(19) United States
(12) Patent Application Publication
(10) Pub. No.: US 2021/0290491 A1
(43) Pub. Date: Sep. 23, 2021

(54) CHAMBERED DISPENSING DEVICES AND METHODS

(71) Applicants: Eric Campos, Fullerton, CA (US); Jennifer Devine Filice, Salinas, CA (US)

(72) Inventors: Eric Campos, Fullerton, CA (US); Jennifer Devine Filice, Salinas, CA (US)

(21) Appl. No.: 17/340,840

(22) Filed: Jun. 7, 2021

Related U.S. Application Data
(63) Continuation of application No. 16/850,594, filed on Apr. 16, 2020.
Application No. 16/850,594 is a continuation-in-part of application No. 16/160,673, filed on Oct. 15, 2018, now Pat. No. 10,918,574.
Application No. 16/850,594 is a continuation-in-part of application No. 16/712,264, filed on Dec. 12, 2019.
Application No. 16/712,264 is a continuation-in-part of application No. 29/690,976, filed on May 13, 2019.
(60) Provisional application No. 62/707,006, filed on Oct. 16, 2017.

(51) Int. Cl. A61J 7/00; G06F 3/0484; G16H 20/13

(52) U.S. Cl. CPC A61J 7/0076; G06F 3/04847; A61J 7/0053; A61J 7/0046; G16H 20/13

(57) ABSTRACT

Chambered dispensing devices and methods for dispensing an amount of a preparation involve the use of a handheld dispensing system having a connecting body housing one or more preparation vials, a delivery spout, and a computer system or microprocessor controlled circuit that controls dispensing of the preparation(s), optionally in combination with a mobile application that allows an individual or user to interact with the chambered dispensing device via a mobile computing device, such as a smart phone. A mobile application can provide a graphic user interface that facilitates operation of the chambered dispensing device by the individual or user, optionally via dispensing device keypad buttons, to dispense micro-dose amounts of primary and secondary cannabinoids or other preparations or substances.

### CROSS-REFERENCES TO RELATED APPLICATIONS

This application is a continuation-in-part of U.S. patent application Ser. No. 16/160,673 filed Oct. 15, 2018, which claims the benefit of U.S. Provisional Patent Application No. 62/707,006 filed Oct. 16, 2017. This application is also a continuation-in-part of U.S. patent application Ser. No. 16/712,264 filed Dec. 12, 2019. The entire content of each of the above patent applications is incorporated herein by reference.

### BACKGROUND OF THE INVENTION

Dispenser devices are used to dispense any of a variety of materials to a user. Commonly known dispenser devices can be operated to deliver Cannabis and other plant preparations to a user. Although these devices can be helpful in providing users with delivered amounts of such preparations, still further improvements are desired. Embodiments of the present invention provide solutions to at least some of these outstanding needs.

### BRIEF SUMMARY OF THE INVENTION

Embodiments of the present invention encompass systems, devices, and methods for dispensing a preparation for consumption or use by a patient or individual. Exemplary preparations include beauty and cosmetic preparations, medical preparations, culinary preparations, homeopathic preparations, whole plant preparations, plant extract preparations, medicament preparations, and the like. In some cases, a preparation may be provided as a fluid. In some cases, a preparation may include a medicament. In some cases, a preparation may include a Cannabis oil, tincture, extract, derivative, and/or constituent. Exemplary devices may be configured to dispense any of a variety of preparations, such as fluids, gels, pastes, liquids, nanoemulsions, and the like. As used herein, the term “preparation” is intended to include flowable materials that can be dispensed using the disclosed devices and methods. In some embodiments, methods, systems, and devices can be used for the oral or sublingual administration of a Cannabis preparation to a user or individual. In some cases, a dispensing device can be configured to dispense micro-dose amounts of Cannabis constituents and/or other compounds or molecules. According to some embodiments, a dispenser device can include two or more function assemblies that together form a full assemblage of a dispenser device. According to some embodiments, a dispenser device may be provided in a reusable configuration.

In one aspect, embodiments of the present invention encompass a mobile computing device for displaying operational instructions for a chambered dispensing device. Exemplary mobile computing devices can include a screen, a processor, an electronic storage location operatively coupled with the processor, and processor executable code stored on the electronic storage location and embodied in a tangible non-transitory computer readable medium. The processor executable code, when executed by the processor, can cause the processor to generate a graphical user interface on the screen, the graphical user interface having a first input that receives a desired micro-dose amount of a first cannabinoid, a second input that receives a desired micro-dose amount of a second cannabinoid, and an output that displays a first operational instruction for the chambered dispensing device corresponding to the first cannabinoid and a second operational instruction for the chambered dispensing device corresponding to the second cannabinoid. In some cases, the first operational instruction includes a setting number for the chambered dispensing device. In some cases, the setting number corresponds to a setting on the chambered dispensing device that can be adjusted with up and down keypad buttons of the chambered dispensing device. In some cases, the graphical user interface further comprises a third input that receives a vial size corresponding to the first cannabinoid or the second cannabinoid. In some cases, the graphical user interface further includes a third input that receives a product name corresponding to the first cannabinoid or the second cannabinoid. In some cases, the product name includes a Cannabis strain name, a tincture formula name, or a custom oil extract name. In some cases, the graphical user interface further includes a third input that receives a product type corresponding to the first cannabinoid or the second cannabinoid. In some cases, the product type includes a brand name or a manufacturer name. In some cases, the graphical user interface further includes a third input that receives a product size corresponding to the first cannabinoid or the second cannabinoid. In some cases, the first cannabinoid is delta-9-tetrahydrocannabinol and the second cannabinoid is cannabidiol. In some cases, the mobile computing device is a smart phone.

In another aspect, embodiments of the present invention encompass a computer program product for instructing a user of a chambered dispensing device. The computer program product can be embodied on a non-transitory tangible computer readable medium. The computer program product can include computer-executable code for generating a graphical user interface on a screen of a mobile computing device. The graphical user interface can include a first input that receives a desired micro-dose amount of a first cannabinoid, a second input that receives a desired micro-dose amount of a second cannabinoid, and an output that displays a first operational instruction for the chambered dispensing device corresponding to the first cannabinoid and a second operational instruction for the chambered dispensing device corresponding to the second cannabinoid. In some cases, the first operational instruction includes a setting number for the chambered dispensing device. In some cases, the setting number corresponds to a setting on the chambered dispensing device that can be adjusted with up and down keypad buttons of the chambered dispensing device. In some cases, the graphical user interface further comprises a third input that receives a vial size corresponding to the first cannabinoid or the second cannabinoid. In some cases, the graphical user interface further includes a third input that receives a product name corresponding to the first cannabinoid or the second cannabinoid. In some cases, the product name includes a Cannabis strain name, a tincture formula name, or a custom oil extract name. In some cases, the graphical user interface further includes a third input that receives a product type corresponding to the first cannabinoid or the second cannabinoid. In some cases, the product type includes a brand name or a manufacturer name. In some cases, the graphical user interface further includes a third input that receives a product size corresponding to the first cannabinoid or the second cannabinoid. In some cases, the first cannabinoid is delta-9-tetrahydrocannabinol and the second cannabinoid is cannabidiol. In some cases, the mobile computing device is a smart phone.

In some cases, a dispenser device can be configured to be reusable, and after a desired amount of preparation has been dispenses from a single vial, the used vial can be removed from the dispenser device and replaced with a new vial that is loaded with a Cannabis preparation. In some cases, a dispenser device can be used for any other type of fluid, liquid, paste, or material that is dispensed, for example in a small volume such as a liquid volume, or for example in a desired amount, in a consistent fashion, or in an exact or precise amount. In some cases, a dispenser device can be configured for use in dispensing an amount of preparation without requiring the use of a needle (e.g. tissue-piercing injection needle). In some cases, a dispenser device can be configured for use in dispensing a liquid or paste by expelling the liquid or paste in fixed amounts that are measured and are repeatable. An exemplary dispenser device may include an exterior housing having one or more component parts. A dispenser device may also include a serviceable cartridge or vial assembly. A vial assembly may include a vial, a displaceable stopper disposed within the vial, and an amount of preparation contained within the vial. A vial may be coupled with a dispenser tip. In some cases, a housing component may be configured to receive a cartridge or vial. In some cases, a housing component may include a control assembly, which may include mechanical and/or electronic mechanisms for providing motive force for the displacement of a stopper within a vial (e.g. as disclosed in previously incorporated U.S. patent application Ser. No. 16/712,264 filed Dec. 12, 2019). In some cases, a dispenser device includes a protective cap. In some cases, a vial assembly can be provided as a refillable vial assembly. In some cases, a vial assembly can be provided as a replaceable vial assembly. In some cases, a control assembly may include a presetting mechanism that allows a user to preset an amount of preparation (e.g. fluid, liquid, paste, gel, or the like) to be dispensed by the dispenser device. In some cases, a control assembly may include an initiating mechanism that allows a user to initiate the dispensing of an amount of preparation (e.g. fluid, liquid, paste, gel, or the like) from the dispenser device. In some cases, an initiating mechanism may be a dispense button. In some cases, a dispenser device can include a communication and/or control mechanism whereby a user can communicate with and/or control the device via radio, inductive, or light signals, Bluetooth technology, devices, or energy.

### BRIEF DESCRIPTION OF THE DRAWINGS

FIG. 1 depicts aspects of a dispensing device, according to embodiments of the present invention.

FIG. 2 illustrates aspects of a dispensing device, according to embodiments of the present invention.

FIG. 3 illustrates aspects of a dispensing device, according to embodiments of the present invention.

FIG. 4 depicts aspects of a dispensing device, according to embodiments of the present invention.

FIG. 5 shows aspects of a dispensing device, according to embodiments of the present invention.

FIG. 6 shows aspects of a dispensing device, according to embodiments of the present invention.

FIG. 7 depicts aspects of a dispensing device, according to embodiments of the present invention.

FIG. 8 illustrates aspects of a dispensing device, according to embodiments of the present invention.

FIG. 9 shows aspects of a dispensing device, according to embodiments of the present invention.

FIG. 10 shows aspects of a graphic user interface, according to embodiments of the present invention.

FIG. 11 shows aspects of a graphic user interface, according to embodiments of the present invention.

FIG. 12 shows aspects of a graphic user interface, according to embodiments of the present invention.

FIG. 13 shows aspects of a graphic user interface, according to embodiments of the present invention.

FIG. 14 shows aspects of a graphic user interface, according to embodiments of the present invention.

FIG. 15 shows aspects of a graphic user interface, according to embodiments of the present invention.

FIG. 16 shows aspects of a graphic user interface, according to embodiments of the present invention.

FIG. 17 shows aspects of a computer system, according to embodiments of the present invention.

FIG. 18 depicts aspects of a dispensing device, according to embodiments of the present invention.

FIG. 19 depicts aspects of a dispensing device, according to embodiments of the present invention.

FIGS. 20A and 20B depict aspects of a dispensing device, according to embodiments of the present invention.

FIG. 21 depicts aspects of a dispensing device, according to embodiments of the present invention.

FIG. 22 depicts aspects of a dispensing device, according to embodiments of the present invention.

FIG. 23 depicts aspects of a dispensing device, according to embodiments of the present invention.

FIGS. 24A and 24B depict aspects of a dispensing device, according to embodiments of the present invention.

FIG. 25 depicts aspects of a dispensing device, according to embodiments of the present invention.

FIG. 26 depicts aspects of a dispensing device, according to embodiments of the present invention.

FIGS. 26A, 26B, 26C, and 26D illustrate aspects of graphic user interfaces, according to embodiments of the present invention.

FIGS. 27A, 27B, 27C, and 27D illustrate aspects of graphic user interfaces, according to embodiments of the present invention.

FIGS. 28A and 28B illustrate aspects of graphic user interfaces, according to embodiments of the present invention.

FIGS. 29A, 29B, and 29C illustrate aspects of graphic user interfaces, according to embodiments of the present invention.

FIGS. 30A and 30B illustrate aspects of graphic user interfaces, according to embodiments of the present invention.

FIGS. 31A and 31B illustrate aspects of graphic user interfaces, according to embodiments of the present invention.

FIGS. 32A and 32B illustrate aspects of graphic user interfaces, according to embodiments of the present invention.

### DETAILED DESCRIPTION OF THE INVENTION

In the Summary above and in this Detailed Description, and the Claims below, and in the accompanying Drawings, reference is made to particular features of embodiments of the present invention. It is to be understood that the disclosure of embodiments of the present invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in embodiments of the invention generally.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

According to some embodiments, the term “at least” followed by a number can be used herein to denote the start of a range including that number (which may be a range having an upper limit or no upper limit, depending on the variable being defined). For example, “at least 1” can mean 1 or more than 1. According to some embodiments, the term “at most” followed by a number can be used herein to denote the end of a range, including that number (which may be a range having 1 or 0 as its lower limit, or a range having no lower limit, depending upon the variable being defined). For example, “at most 4” can mean 4 or less than 4, and “at most 40%” can mean 40% or less than 40%. According to some embodiments, when, in this specification, a range is given as “(a first number) to (a second number)” or “(a first number)-(a second number),” this can mean a range whose limits include both numbers. For example, “25 to 100” can mean a range whose lower limit is 25 and upper limit is 100, and includes both 25 and 100.

Embodiments of the present invention encompass systems and methods for dispensing or administering a precise, customized dose of one, two, or more preparation constituents. In exemplary embodiments, dispensing devices and methods can be used to deliver or dispense one or more preparations, which may be provided as fluid or liquid compounds or agents, from isolated reservoirs or chambers. In some cases, dispensing devices may include one, two, three, or more separate chambers or reservoirs. Embodiments encompass dual chambered modules having a separate reservoir or chamber housing a single preparation or liquid and at least a second separate reservoir or chamber housing another type of preparation of liquid. The separate chambers can enable the user with an option of micro dosing one or more individual cannabinoids to a custom level as desired by the user's preferences or as directed by a health care professional. In some cases, the compounds may be available in two or more chambers each containing independent compounds or mixtures. In some cases, a chamber can be provided with a pre-mixed preparation. In some embodiments, a chamber can be provided with co-formulated multiple molecular compounds. In exemplary embodiments, a user can control the dosing of one or more constituents of one or more preparations in order to tailor a final mixture or preparation to a specific desired or therapeutic profile. In some cases, such customized dispensing techniques can provide a user with a desired or therapeutic response. In some cases, such customized dispensing techniques can be tailored for a specific target user or patient group.

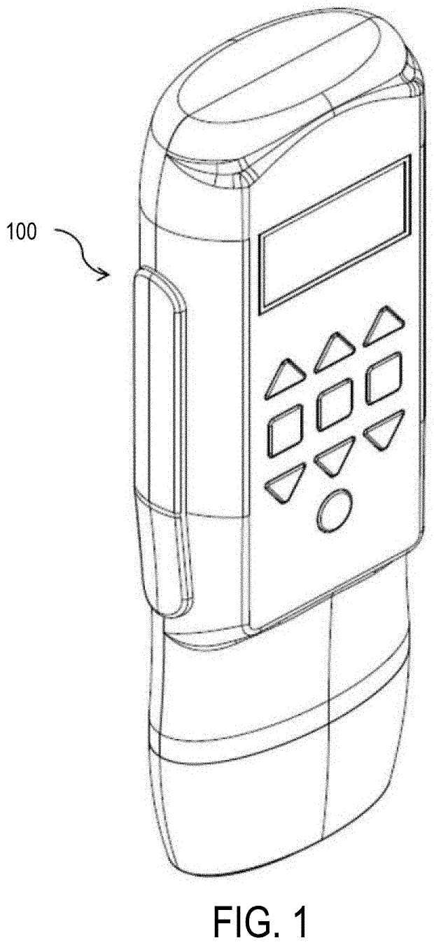

Turning now to the drawings, FIG. 1 depicts aspects of a chambered dispensing device 100 according to embodiments of the present invention. In some cases, dispensing device 100 may be provided as a multi-chamber dispensing device. In some cases, dispensing device 100 can include three compound or preparation delivery vials.

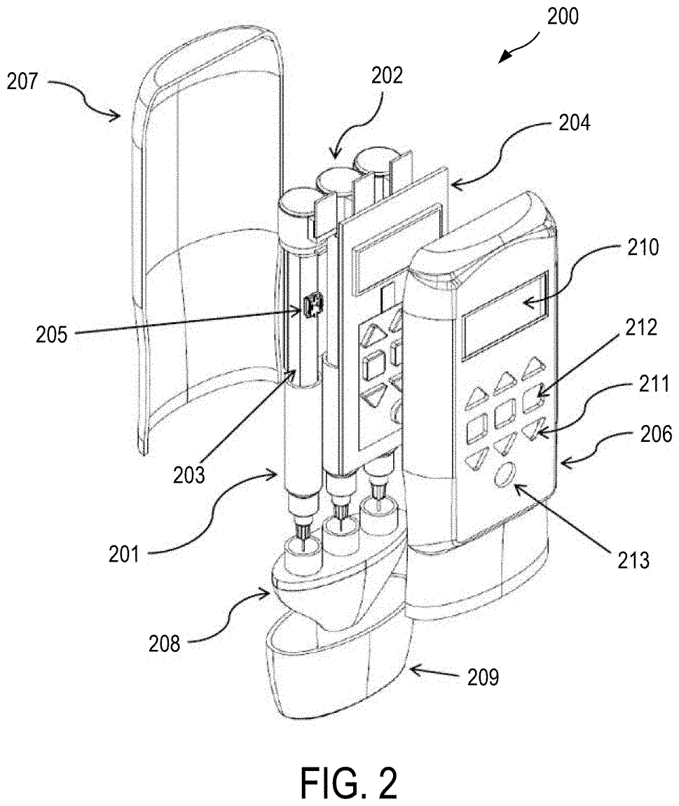

FIG. 2 depicts aspects of a chambered dispensing device 200 according to embodiments of the present invention. As shown here, the dispensing device 200 can be a multi-chamber dispensing device, having multiple compound or preparation delivery vials or chambers 201. For example, dispensing device 200 may include three preparation vials 201. The vials 201 can be connected to or otherwise in operational engagement with stepper motors 202 (e.g. one vial in connectivity with one respective stepper motor). The dispensing device 200 can also include a plunger 203 at the end of, or otherwise in operational engagement with, a respective stepper motor 202. In use, a stepper motor can operate to actuate a plunger, so as to dispense an amount of a preparation from a vial. Dispensing device 200 also includes a circuit board assembly or computing system 204, which may include a microprocessor controlled circuit, and may be in operational connectivity with one or more stepper motors, and other components of the dispensing device 200 described herein. Dispensing device 200 also includes a charging port 205, a front housing 206, a rear housing 207, a dispensing spout 208, an end cap 209, an indicator screen 210, adjustment UP and DOWN keypad buttons 211, a dispensing button 212, and an ON/OFF button 213. In some embodiments, the dispensing spout 208 can be configured to receive one or more amounts of expelled preparation from one or more of the vials (e.g. via one or more inlet conduits), and to dispense the one or more amounts of preparation (e.g. via an outlet conduit). As shown here, dispensing spout 208 includes three inlet conduits and one outlet conduit.

The front housing 206 and rear housing 207 can cover the circuit board assembly or computing system 204 and the stepper motors 202, and can leave the tips of vials 201 exposed so as to facilitate a simple vial replacement procedure. The end cap 209 can cover the cylindrical shaped vials 201 after each use (e.g. dispensing of preparation). In some cases, the front and rear housing can form at least part of a connecting body, and the connecting body may include a location or holding container for one, two, three, or more separate reservoirs or chambers.

Externally actuation of the ON/OFF button 213 can operate to control the power of the dispensing device 200, which in some embodiments may time out after 30 seconds of non-use. The indicator screens 210 can be configured to show the amount of the dose as chosen by the user via actuation of the UP and DOWN keypad buttons 211. The dispensing button 212 can operate to activate the dispensing process which can precisely control one or more chambers of the preparation(s).

A user can select the preparation dosage via one or more of the UP/DOWN keypad buttons 211, and setting the dose of a preparation by a user can give an individual or patient more reliable control over a desired or therapeutic outcome of micro-dosed treatment or therapy. In some embodiments, a combined variable dose treatment or therapy may be preferable over a single dose treatment or therapy. In some embodiments, a preparation or compound dispensing interface 208 can include a hollow spout for the delivery dispensing of fluid or liquid preparations or compounds. In some embodiments, the dispensing device 200 may include a touch pad interface for simple UP and DOWN control of the dispensing or dosing device.

The circuit board assembly or computing system 204 can be in operational connectivity with one or more of the stepper motor(s) 202, indicator screen 210, adjustment UP and DOWN keypad buttons 211, dispensing button 212, and/or ON/OFF button 213. In use, the computing system 204 can receive input signals or data from adjustment UP and DOWN keypad buttons 211, dispensing button 212, and/or ON/OFF button 213, and can provide output signals or data to one or more of the stepper motor(s) 202 and/or indicator screen 210.

A multi-chamber medicament dispensing device 200 can have medicament modules/vials 201, and a delivery spout 208. The dispensing device 200 can be controlled electronically. In some cases, the modules or vials 201 may be interchanged and replaced with various medicaments. In some cases, the modules or vials 201 may provide a precise volume of medicaments. In some cases, the modules or vials 201 may provide a precise volume of cannabis-derived medicaments. In some cases, the dispensing device 200 is configured to be electronically tracked, mechanical, and non-motorized.

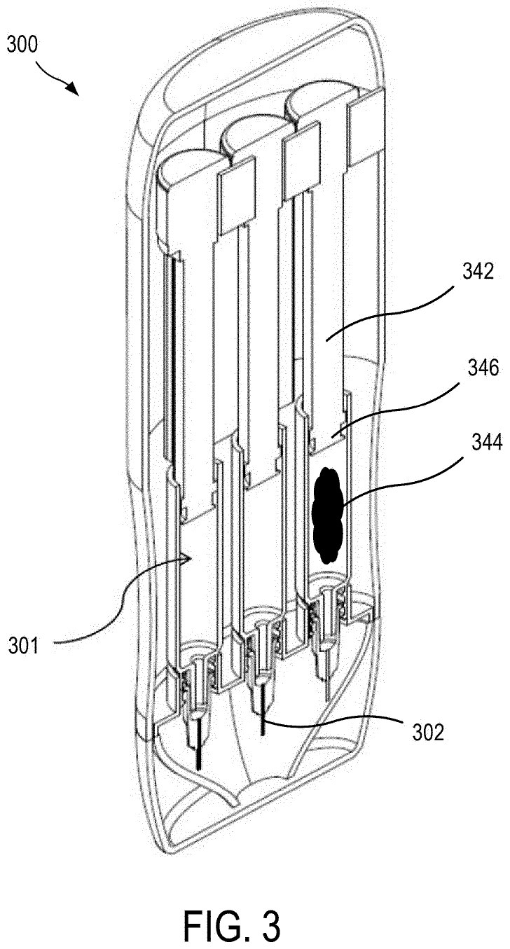

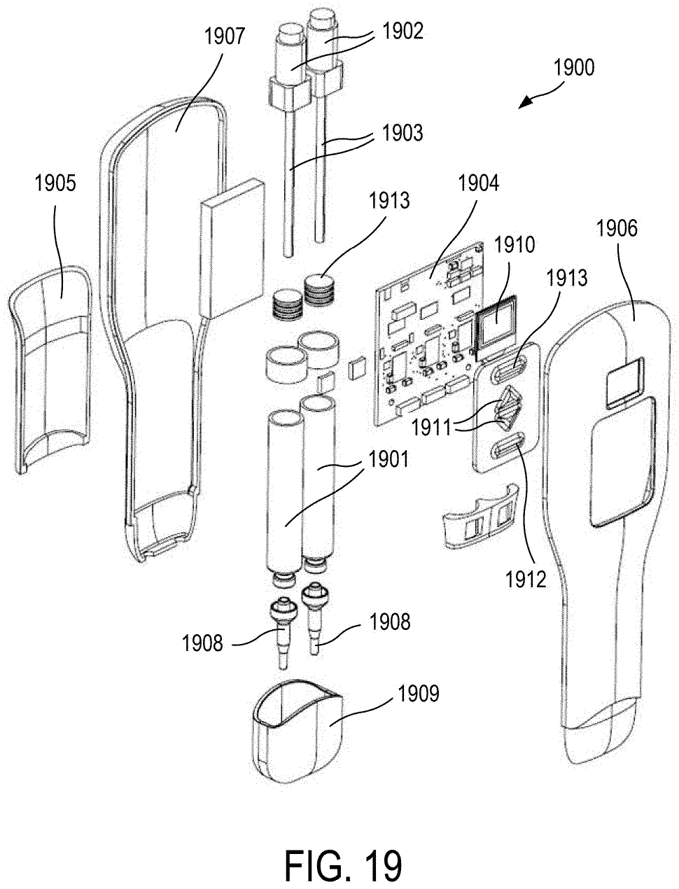

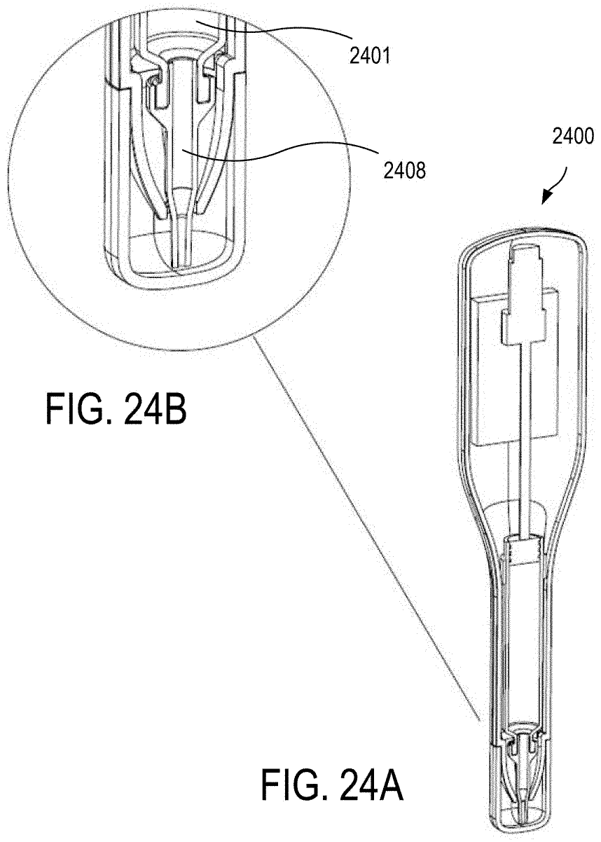

FIG. 3 depicts aspects of a chambered dispensing device 300 according to embodiments of the present invention. As shown here, the dispensing device 300 can be a multi-chamber dispensing device, having multiple (e.g. three) compound or preparation delivery vials or chambers 301. According to some embodiments, actuation of a stepper motor can move a plunger 342 or other actuating mechanism which in turn drives a stopper 346 within the vial 301 from a proximal location within the vial 301 to a more distal location within the vial 301, thus dispelling an amount of a preparation 344 from the vial. In some cases, an actuating mechanism, as discussed elsewhere herein, may include a shaft, a piston, a plunger, or the like. In some embodiments, the stopper 346 is made of silicone. It has been surprisingly discovered that silicone is an unexpectedly advantageous material to use in fabrication of the stopper 346, because it tends not to absorb components of a Cannabis preparation. Silicone is also advantageous because it is not permeable. Additional and alternative aspects of a stopper are described elsewhere herein, for example with reference to FIGS. 19 and 24.

According to some embodiments, a dispensing device 300 may have one or more dispenser tips 302 having any of a variety of desired sizes and/or gauges. Additional and alternative aspects of a dispenser tip are described elsewhere herein, for example with reference to FIGS. 19 and 24. In some cases, parameters of a dispenser tip or the capacity of a dispenser tip can be selected based on a particular viscosity range of the preparation to be dispensed. For example, the parameters of a dispenser tip and/or the capacity of a dispenser tip can be selected so as to achieve a desired or optimal flow for the preparation to be dispensed. In some cases, the size and/or gauge may be selected based on the consistency or viscosity of preparation to be dispensed. For example, with a more viscous preparation material, which may be a Cannabis dab material, a Cannabis gel material, or a Cannabis honey material, a dispenser tip with a wider gauge distal nozzle may be used (e.g. as compared to a dispenser tip for use with a less viscous Cannabis oil preparation material). Relatedly, with more viscous preparation materials, it may be advantageous to use a dispenser tip having a metal (e.g. stainless steel) distal nozzle instead of a plastic distal nozzle. Dispenser tips having metal distal nozzles can also be used with preparation materials which may benefit from heating prior to dispensing, such as Cannabis dab preparations.

According to some embodiments, a vial or chamber 301, which may be provided as liquid-holding or paste-holding vial in some embodiments, can employ a stopper 346, which may be a rubber piston or a silicone piston, for example, to both seal a proximal opening of the vial 301, and when pressed further into vial 301 in a distal direction, to create pressure and/or force that in turn causes an amount of a preparation (e.g. liquid) to be expelled from the vial 301 into dispenser tip 302, and thereafter out of dispensing tip 302.

According to some embodiments, the stopper 346 may include a plurality of annular rings which operate to create a plurality of seals against the vial, and the annular rings are sized so that the stopper 346 can slide within the interior chamber of the vial 301. In some embodiments, stopper 346 is made of rubber. It is understood that stopper 346 may be made of any suitable material, as a means of example but not of limitation, including plastics, rubbers, silicones, fluoropolymers; or any combination thereof. In some cases, stopper 346 can be provided as a springy metal ring. In some embodiments, stopper 346 is made of silicone. According to some embodiments, a stopper 346 made of silicone and having a plurality of rings (e.g. four rings) can provide certain operational advantages. For example, some known pen devices use an aluminum and rubber seal crimped unto the neck of a vial to create a vacuum seal of the liquid contained therein. Each time an operator needs to use such a pen, the operator must screw on a new needle that pierces through the rubber seal, so as to enable the delivery of the liquid. Such pen devices typically require a stiff rubber stopper having a sufficiently wide diameter, so as to ensure that a high amount of pressure is retained inside the vial at all times. In contrast, some embodiments of the present invention include a stopper made of a softer silicone material with a smaller diameter, optionally with rings, that will give and move easier. By using a softer silicone material with a smaller diameter for the stopper, it is possible to ensure that the pressure is not excessively high which would cause leakage, and that the stopper provides a sufficiently tight seal which facilitates the dispensing of liquids quickly and accurately. In some embodiments, a stopper includes a number of rings or ribs (e.g. 4) which operate to provide a slight grip on the inner wall of the vial (e.g. a glass vial) without sticking to the vial at any point.

It has surprisingly been discovered that using silicone as a material for the stopper 346 (instead of, for example, butyl rubber) can be particularly advantageous for various reasons. It has been discovered that a silicone stopper slides easily within a glass vial, whereas a rubber stopper may exhibit stickiness to the vial. Moreover, a silicone stopper has been observed to exhibit less leaking and to retain a better seal, as compared with a butyl rubber stopper. It was also surprisingly discovered that adding more annular rings (e.g. for a total of four rings) to the stopper resulted in improved performance, by helping to reduce any stickiness between the stopper and the vial.

Moreover, it was unexpectedly discovered that by using silicon to fabricate the stopper, the stopper did not require additional processing techniques such as coating or curing, in order to obtain the desired performance. It was surprisingly discovered that silicone did not absorb constituents of the preparation contained within the vial (e.g. Cannabis constituents such as cannabinoids), whereas unwanted absorption was observed with a butyl rubber stopper. Moreover, it was surprisingly discovered that when using a silicone stopper, Cannabis constituents such as cannabinoids did not deteriorate when they were stored within the vial, as compared with the deterioration which was observed when a preparation containing Cannabis constituents was stored in a vial assembly that included a butyl rubber stopper. When comparative testing between a silicone stopper and a butyl rubber stopper was performed, it was observed that the active cannabinoid content within a vial assembly having a silicone stopper remained more consistent or at higher levels, as compared to the active cannabinoid content within a vial assembly having a butyl rubber stopper. In some cases, the testing was performed over the course of a week.

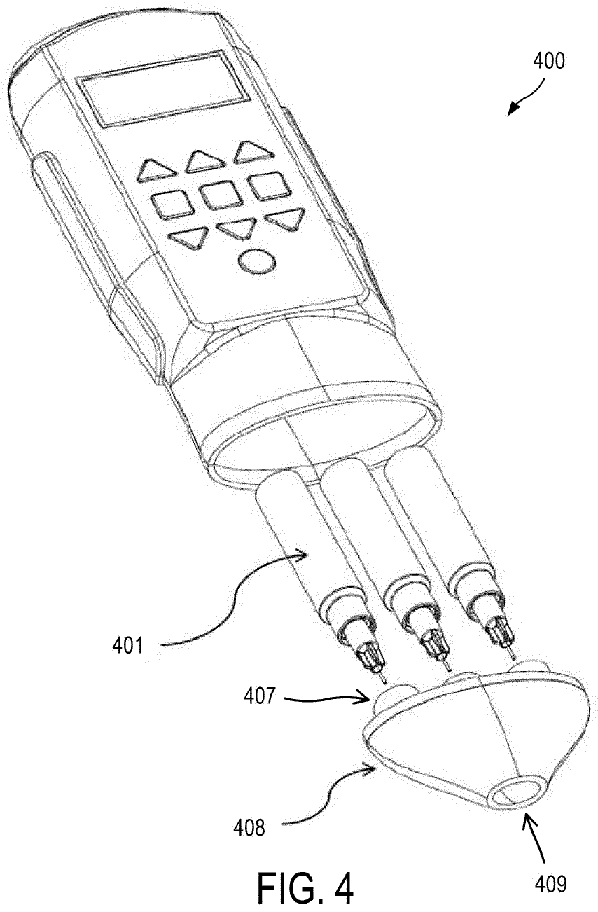

FIG. 4 depicts aspects of a chambered dispensing device 400 according to embodiments of the present invention. As shown here, the dispensing device 400 can be a multi-chamber dispensing device, having multiple (e.g. three) compound or preparation delivery vials or chambers 401. Individual vials or chambers can have or be in fluid communication with respective spouts, which can deliver preparation to respective inlet conduits 407 of a dispensing spout 408. As shown here, dispensing spout 408 can include three inlet conduits 407 and one outlet conduit 409. Each of the inlet conduits 407 can be in fluid communication with the outlet conduit 409. In some embodiments, the dispensing spout 408 may be screwed onto or otherwise secured in fluid communication with the end of one or more of the vials or chambers 401 for a secure fit that is ready to dispense one or more preparations or contents of the vials. As discussed elsewhere herein, a vial or chamber may include or be in operate association with a stopper. In some cases, a stopper can include or be constructed of a material such as TPE (Thermoplastic Elastomers), Liquid Silicone Rubber (LSR), natural rubbers, Low-density Polyethylene (LDPE), Linear low-density Polyethylene (LLDPE), or combinations thereof.

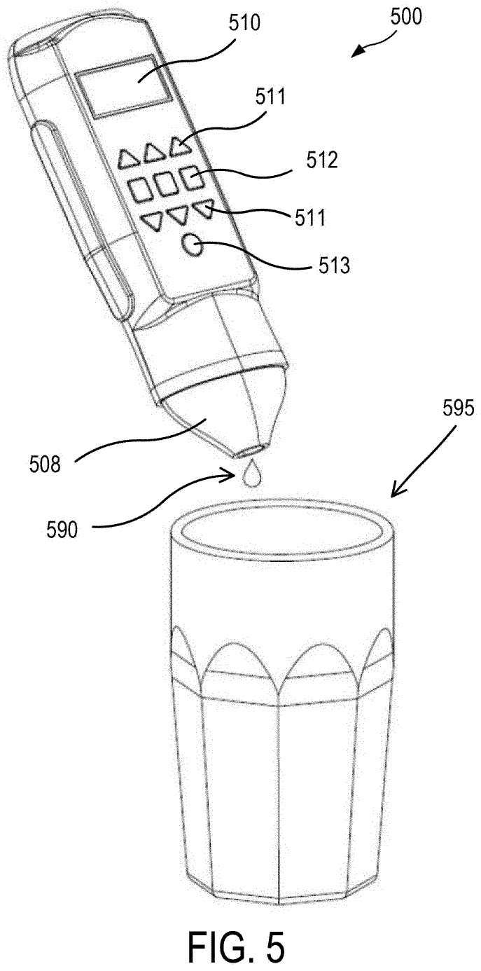

FIG. 5 depicts aspects of a chambered dispensing device 500 according to embodiments of the present invention. As shown here, dispensing device 500 can be used to dispense an amount of preparation 590 therefrom, for example into a container or receptacle 595 such as a drinking glass. In some cases, a dispensing device can be used to dispense an amount of preparation directly on the tongue or into the mouth of an individual. In some cases, a dispensing device can be used to dispense an amount of preparation directly on the tongue or into the mouth of a pet or a mammal. In some cases, a dispensing device can be used to dispense an amount of preparation into a container of any kind, on or into food or any other consumable item or material, or onto the skin of a recipient (e.g. individual person, pet, or mammal). In some cases, a dispensing device can be used to dispense an amount of preparation on or into any desired location of a recipient. For example, the dispensing device can be used to dispense an amount of preparation for mucosal delivery (e.g. buccal, ocular, nasal, vaginal, or rectal), ocular delivery, or transdermal delivery. Dispensing device 500 may include a dispensing spout 508. Dispensing device 500 can also include UP and DOWN dose control buttons 511 and a display 510, such as a numbered LED display or indicator screen. Further, dispensing device can include one or more dispensing or GO buttons 512, and an ON/OFF button 513. In operations, a dose for dispensing from the dispensing device 500 can be set using one or more of the dose setter buttons 511, e.g. by selecting an appropriate number of units or incremental amounts of preparation. Dispensation of the fluid or liquid compound or preparation may then be achieved by dispensing the preparation (e.g. water soluble liquid contents) via activation of the GO button 512 on device 500. In some embodiments, the dose buttons 511 are operably connected to a printed circuit board (PCB) assembly or computing system (e.g. computing system 204 depicted in FIG. 2) that engages one or more stepper motors to push on reservoirs of each vial containing the fluid or liquid preparations.

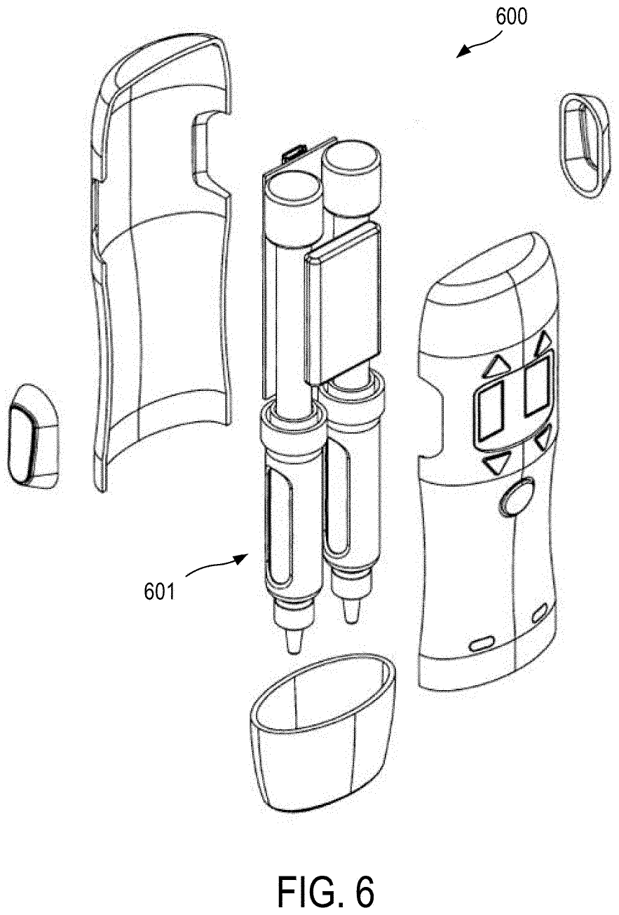

FIG. 6 depicts aspects of a chambered dispensing device 600 according to embodiments of the present invention. As shown here, the dispensing device 600 can be a multi-chamber dispensing device, having multiple (e.g. two) compound or preparation delivery vials or chambers 601. As further discussed elsewhere herein, in addition to the dual chamber dispensing device embodiment depicted in FIG. 6 and the triple chamber dispensing device embodiment depicted in FIG. 2, the instant disclosure encompasses single chamber dispenser embodiments, such as that which is depicted in FIG. 24. Hence, dispensing device embodiments can be provided in any of a variety of single, dual, and other multi-chamber digital devices.

With returning reference to FIG. 6, in some embodiments, dispensing device 600 includes two stepper motors with actuators, a plunger at the end of each actuator, a printed circuit board assembly, a USB charging port, a battery pack, a front housing, a rear housing, two cartridge or vial holders, glass or plastic replaceable vials or cartridges, a dispensing spout at the end of each vial or cartridge, an end cap, one or more indicator screens, one or more LED indicator lights, adjustment UP and DOWN keypad buttons, a dispense GO button, and an ON/OFF button. In some embodiments, the front and rear housings can cover the printed circuit board (PCB) assembly. In some embodiments, the front and rear housings can leave vial holders exposed so as to facilitate vial replacement. The end cap can cover the vial holders, which may be cylindrical shaped, and liquid vials after each use. In use, externally the ON/OFF button can control the power of the device, and the power of the device can time out after 30 seconds of non-use. In some embodiments, an indicator screen can show the amount of the dose selected with the UP and DOWN keypad buttons. In some cases, the dispensing GO button can activate the dispensing process, which precisely controls dispensing of preparation (e.g. liquid compounds) from one or both chambers of the vials. In some cases, LED indicator lights can provide an indication to the user when the liquid or fluid doses have been delivered, for example by turning from a green color to a red color.

Embodiments of the present invention encompass systems (such as dispensing device 600) and methods for administering a custom variable dose of a liquid compound (e.g. primary cannabinoid compound) and a variable dose of another liquid compound (e.g. secondary cannabinoid compound, or other compound) through a multiple-output/multiple-chamber or liquid compound dispensing interface. A user can select a dose of a primary and/or secondary compound, which can provide the user with reliable control over a desired or therapeutic outcome of a micro dosed treatment or therapy. In some embodiments, a combined variable dose treatment or therapy may be preferable over a single dose treatment or therapy. In some embodiments, a preparation or compound dispensing interface can include a hollow spout for the delivery dispensing of fluid or liquid preparations or compounds. In some embodiments, the dispensing device may include a touch pad interface for simple UP and DOWN control of the dispensing or dosing device 600.

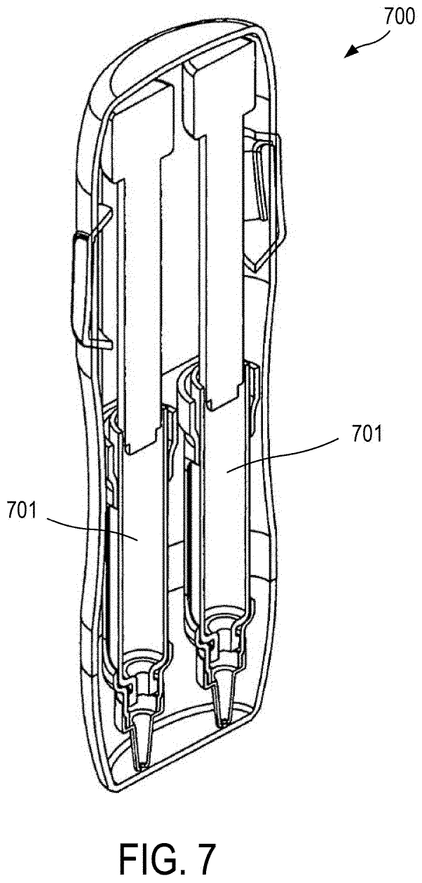

FIG. 7 depicts aspects of a chambered dispensing device 700 according to embodiments of the present invention. As shown here, the dispensing device 700 can be a multi-chamber dispensing device, having multiple (e.g. two) compound or preparation delivery vials or chambers 701. According to some embodiments, to connect the liquid component vials with other components of the dispensing device, the glass or plastic vials can be inserted into an actuator assembly and locked in with a vial holder. In some cases, a dispensing spout can be screwed onto the end of a vial holder, for a secure fit, so that the dispensing device 700 is ready to dispense liquid compounds.

According to some embodiments, an individual vial can include or be used with a respective stopper, which may be constructed of or include a rubber materials. The stopper can operate to seal (e.g. pressure seal) the liquid content of the vial. In some cases, an end cap with a pierceable membrane can be used with the delivery spout. In some cases, a pierceable membrane can include or be constructed of a material that is broadly inert when placed in long term contact with liquid compounds, and can offer good performance with respect to leachable and/or extractable liquids. In some cases, a membrane can include or be constructed of a material such as TPE (Thermoplastic Elastomers), Liquid Silicone Rubber (LSR), natural rubbers, Low-density Polyethylene (LDPE), Linear low-density Polyethylene (LLDPE), or combinations thereof.

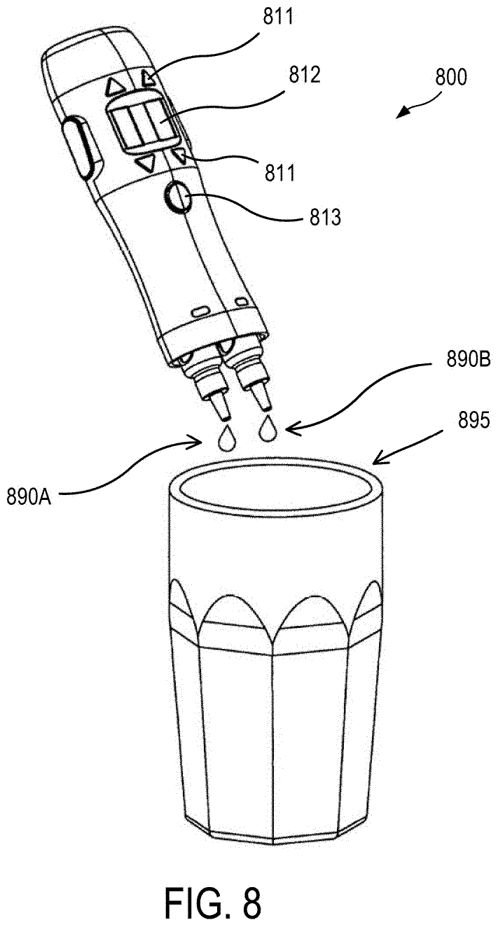

FIG. 8 depicts aspects of a chambered dispensing device 800 according to embodiments of the present invention. As shown here, the dispensing device 800 can be a multi-chamber dispensing device, having multiple (e.g. two) compound or preparation delivery vials or chambers 801. The device 800 can be used with the vials (e.g. fluid or liquid compound vials) to spray or dispense fluid or liquid into a beverage container. The embodiment depicted in FIG. 8 provides an exemplary layout of a housing face view of a dispensing device showing the UP and DOWN dose control buttons with an indicator display. As shown here, dispensing device 800 can be used to dispense one or more amounts of one or more preparations 890A, 890B therefrom, for example into a container or receptacle 895 such as a drinking glass. In some embodiments, an amount of preparation 890A can include a tincture or preparation containing CBD with very little or no THC. Exemplary dominant CBD tinctures or preparations include 20:1 to 30:1 CBD to THC ratios. In some embodiments, an amount of preparation 890B can include a tincture containing THC with very little or no CBD. Exemplary dominant THC tinctures or preparations include 10:1 to 2:1 THC to CBD ratios. A user can operate the device 800 (e.g. using the UP/DOWN arrow keys 811) so as to designate a desired number of milligrams of THC and/or CBD (e.g. from respective vials or chambers containing the preparations) for dispensing from the device 800. For example, a user can operate the device 800 to dispense an amount of preparation (which may be a combined preparation) having a 1:1 ratio of THC and CBD. Dispensing device embodiments disclosed herein can be configured to dispense other types of materials that are not related to Cannabis, such as beauty and cosmetic preparations, medical preparations, culinary preparations, homeopathic preparations, whole plant preparations, plant extract preparations, medicament preparations, and other preparations discussed elsewhere herein. Dispensing device embodiments disclosed herein can be configured to dispense Cannabis related materials that are not THC or CBD, such as CBN and other cannabinoid preparations discussed elsewhere herein. Dispensing device embodiments disclosed herein can be configured to dispense any other psychoactive/psychotropic and/or non-psychoactive/non-psychotropic materials.

As shown here, dispensing device 800 can include UP and DOWN dose control buttons 811 and a display, such as a numbered LED display or indicator screen. Further, dispensing device 800 can include one or more dispensing or GO buttons 812, and an ON/OFF button 813. In operations, a dose for dispensing from the dispensing device 800 can be set using one or more of the dose setter buttons 811, e.g. by selecting an appropriate number of units or incremental amounts of preparation. Dispensation of the fluid or liquid compound or preparation may then be achieved by dispensing the preparation (e.g. water soluble liquid contents) via activation of the GO button 812 on device 800. In some embodiments, the dose buttons 811 are operably connected to a printed circuit board (PCB) assembly or computing system (e.g. computing system 204 depicted in FIG. 2) that engages one or more stepper motors to push on reservoirs of each vial containing the fluid or liquid preparations.

Dispensing device 800 can be used with liquid compound vials for spraying liquid contents into a beverage container. As shown here, dispenser device 800 can include UP and DOWN dose control buttons and an indicator display. According to some embodiments, a dispenser device 800 can include dose setter buttons, and a dose to be dispensed by the dispensing device can be set or selected by using the dose setting buttons, for example by selecting an appropriate number of units. Dispensing of the liquid compounds can be achieved by dispensing water soluble liquid contents from the dispensing device via activation of a GO button on the device. In some embodiments, the dose buttons can be operably connected with a printed circuit board (PCB) assembly that engages or is in otherwise operational connectivity with each stepper motor, to push on reservoirs of one or more vial containing liquid compounds.

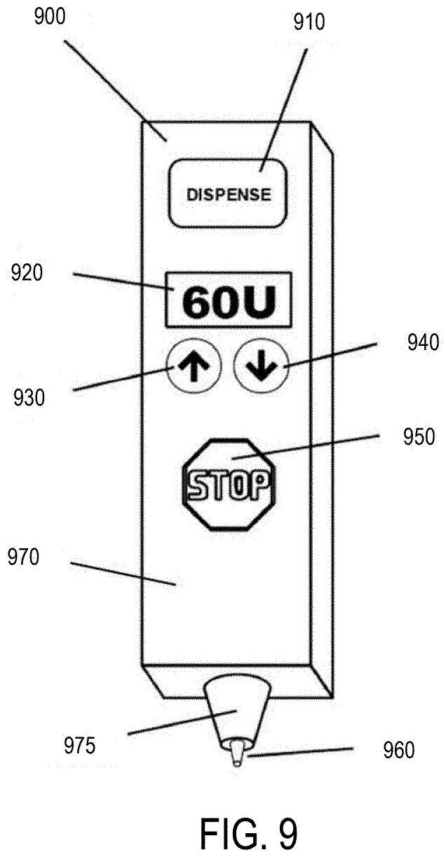

FIG. 9 depicts aspects of a chambered dispensing device 900 according to embodiments of the present invention. Dispenser device 900 can include or incorporate an electronically-controlled mechanism for fixed-amount control and delivery of an amount of a preparation. Conveniently, dispenser device 900 may be sized to be kept in a purse. Dispenser device 900 can include a dispense button 910 that can be used (e.g. actuated or depressed) by an operator to initiate dispensing of a fixed-amount of preparation or liquid once the fixed-amount amount is set by using local fixed-amount setting display 920, or by a linked digital controller. Fixed-Amount selection buttons 930 and 940 can be used with local fixed amount selection display 920, to set the fixed-amount. Stop button 950 can be used to stop a fixed-amount while being dispensed, and to reset the dispenser device 900. A cone 975 of (or coupled with) a housing 970 can be shaped to hold, receive, or otherwise engage an internal cartridge subassembly or vial, as disclosed elsewhere herein. A distal portion of a cartridge subassembly or vial (e.g. which may include an exit port of a dispenser tip 960) can serve as a conduit through which an amount of preparation is dispensed from the dispenser device 900.

In some embodiments, the overall size of the housing 970 allows for an electric motor drive and electronic control system, possibly with a microprocessor or computer system, to be part of or incorporated into the dispenser device 900. In some embodiments, control circuitry of the dispenser device 900 could incorporate radio circuitry or infra-red circuitry to establish a link to a digital controller. For example, but not by means of limitation, such digital controller could be a custom device, smartphone, a tablet, or a computer; and the associated communications may be direct, or thru the internet or other digital or analogue communications medium. In some embodiments, aspects of the dispenser device 900, such as the housing 970, can be configured for use with a stand (e.g. to be held by a stand). Relatedly, in some cases, the dispenser device 900 can be supported by a stand, above a glass or mug, for use in the home or office.

In some cases, any of the dispensing device embodiments disclosed herein may include a biometric security feature (e.g. iris recognition mechanism, fingerprint recognition mechanism, voice recognition mechanism, and the like). Such features can be used in combination with an application software (e.g. mobile app) that can be used to secure the dispenser device, and/or to prevent the dispenser device from dispensing a preparation (e.g. medicament) to a person other than the patient or intended user.

Embodiments of the present invention encompass dispensing devices and related methods of delivering one, two, three, or more preparations or agents from isolated reservoirs using a dispensing device with one, two, three or more separate chambers or reservoirs. Exemplary embodiments encompass single, dual, or multi-chamber modules having a separate reservoirs or chambers containing or housing a preparation and at least a second separate reservoir or chamber housing another preparation. The separate chambers may provide a user an option of micro-dosing each preparation to a custom level according to the user's preference or tolerance or according to the recommendation of a health care professional. The preparation may be available in two or more chambers each containing independent (single molecule compounds) or pre-mixed (co-formulated multiple molecule compounds). Additionally, dispensing device embodiments may be optimized for a specific target user or patient group through control of a dosing and definition of a desired or therapeutic profile.

Users or patients may prefer and/or require cannabinoid and terpene compounds to be delivered in a specific relationship with each other in order to deliver the optimum or desired therapeutic dose. Dispensing devices and methods disclosed herein may be of particular benefit where combination therapy is desirable, but not possible in a single formulation for reasons such as, but not limited to, stability, dose amount, dose control and compromised therapeutic performance and toxicology. Micro-dosing cannabis can involve taking a very measured micro-dose of THC, a psychoactive ingredient, and a measured micro-dose of CBD, a non-psychoactive therapeutic ingredient in cannabis. According to some embodiments, with micro-dosing, users can obtain the maximum benefit from the minimum amount, without becoming overly affected. The development of micro-dosing regimens can involve developing the appropriate minimum dose and developing the appropriate products that will deliver such a dose.

Systems and methods disclosed herein enable a user or individual to adjust or set a dose of one cannabinoid (such as THC) to a custom or desired level and also to adjust or set a dose of a separate cannabinoid or mixture of cannabinoids (such as CBD) to a custom or desired level. Such embodiments can allow a user, healthcare professional, patient, or other individual to have flexibility in titrating bioactive or medicinal compounds up or down with more precision. Embodiments also enable users or individuals to precisely vary the quantity of one or both reservoirs of cannabinoids. For example, one fluid quantity may be varied by changing the programming of the dispensing device. This may be completed by selecting or adjusting a user variable dose or changing the device's “fixed” dose. The second bioactive or medicament quantity may be changed exactly the same way. The user or healthcare professional may then select the most appropriate micro-dosing quantity for a particular treatment regime.

Cannabinoids (e.g. CBD, THC) are hydrophobic substances. Cannabinoids can also be formulated to be water-compatible and appear water-soluble. For example, CBD, THC, and other oils can be made water-compatible if they are formulated as oil-in-water nanoemulsions, which are stable and visually homogeneous oil/water mixtures. Nanoemulsions can be prepared in concentrated forms (tens of mg/ml) that are fully miscible with water and, therefore, appear water-soluble. In addition, nanoemulsions can be made translucent and practically tasteless, which means that they can be mixed into water without compromising its optical clarity or taste. Nanoemulsions are beneficial because they are easily mixed into beverages, translucent, low-taste and safe for consumption, exceptionally high bioavailability, fast onset of action, and stable blood level and high CBD/THC loading capacity. Embodiments of the present invention encompass chambered dispensing devices and methods which incorporate nanoemulsion preparations to provide a precise, high water soluble, and bioavailable product for cannabis consumption.

Embodiments of the present invention encompass modules, systems, and methods that allow for the dose controlled dual or multiple dispensing of medicaments within a single device. In some embodiments, such a system includes two dispensing spouts each individually attached to separate liquid chambers. Each individual chamber can be attached to a stepper motor that is linked to software-driven motor control for precise dispensing of preparation or liquid compounds.

A user can set and dispense a pre-determined dose of preparation or medicaments through one chamber and set a different dose to dispense from the other chamber. In some cases, the dual compound dispenser interface may be programmed to dispense each chamber with a preset micro-dose. The multiple chambered dose setter controls step motors of the device such that a predefined quantity of medicaments is delivered when each chambered dose is set and dispensed through the dual compound dispense interface.

By defining a desired or therapeutic relationship between the individual cannabinoid compounds a delivery device can help ensure that a patient/user receives the optimum desired or therapeutic combination dose from a multi-cannabinoid compound device without the inherent risks associated with single dose devices. The cannabinoids and terpenes can be water soluble fluids that make it easy to dispense, dissolve, and mix into beverage of choice.

Systems and methods disclosed herein can be of particular benefit to users or patients with THC sensitivity and who are not sure of what dose will work best for their condition. Giving the user the ability to titrate up or down in micro-doses insures better patient tolerance and desired or therapeutic benefits. Systems and methods disclosed herein can eliminate the need for trial and error single dose consumption that current dropper based systems offer. Systems and methods disclosed herein are also of particular benefit to patients experiencing the fear or phobia associated with THC consumption.

According to some embodiments, a chambered delivery device has replaceable modules or vials and can be used more than once and therefore is multi-use. It is possible to have a selection of different cannabinoid vials/modules for various conditions that could be provided to individuals or prescribed to patients.

According to some embodiments, a chambered delivery system has a mechanical non-motorized stepper feature, wherein both chambers have a mechanical plunger feature that are part of the medicament delivery device.

Embodiments of the present invention encompass systems, devices, and methods for dispensing or administering a precise, customized dose of a preparation or another desired material. Exemplary devices provide the user to select the dosage amount to be dispensed, thus providing the user with reliable control over the outcome of a micro-dosed administration, which may occur in the user themselves, or in another individual such as a patient or consumer. In some embodiments, a device includes a hollow spout for the delivery or dispensing of a preparation, which may include a liquid compound, for example. In some embodiments, a device can include a dose setting and dispensing mechanism that allows the user to control the device in a simple and efficient manner. In some cases, an individual can operate a dispenser device to deliver an amount of a preparation into their own mouth or into another person's mouth, so as to provide an oral or sublingual administration of the preparation. In some cases, an individual can operate a dispenser device to deliver an amount of a preparation into a cup or a container, and then the individual or another person can consume the preparation from the cup or container, so as to provide an oral or sublingual administration of the preparation.

Dispenser device and method embodiments disclosed herein may provide advantages over other delivery modalities, such as vaping, standard tincture droppers, edibles, and the like. Vaping may be inconvenient for public consumption. Standard tincture droppers for sublingual administration may provide uneven or inconsistent drop distributions. Edibles are typically provided in pre-defined amounts (e.g. 2.5 mg dose) which cannot conveniently be subdivided for the administration of smaller dosage amounts. Dispenser device and method embodiments disclosed herein can provide precise and consistent dosages, in any desired amount, in a repeatable fashion, every time. Dispenser device and method embodiments disclosed herein can also provide for the ingestion of whole plant Cannabis oils and tinctures. Dispenser device and method embodiments disclosed herein can provide a user with a discrete and convenient delivery modality which can be carried in a purse or pocket.

Exemplary disposable dispenser devices can be configured to include vials having a 3 milliliter capacity, and to provide a dosing capacity of 0.1 mg to 2.5 mg of Cannabis preparation or constituent(s) per dose or a dispensed fixed-amount. Disposable dispenser devices can be configured to dispense sublingually or into any beverage using a water-soluble Cannabis extracted oil formulation.

Exemplary reusable dispenser devices can be configured to include vials having a 3 milliliter capacity, and to provide a dosing capacity of 0.1 mg to 2.5 mg of Cannabis preparation or constituent(s) per dose or a dispensed fixed-amount. Reusable dispenser devices can be configured to dispense sublingually or into any beverage using a water-soluble Cannabis extracted oil formulation.

Dispenser devices and methods as disclosed herein are well suited for use in dispensing small amounts of preparations such as liquids, pastes, fluids, and other materials, often measured in small portions of milliliters. Exemplary preparations may include lubricating oils, liquid medicaments, a liquid catalyst, water, liquid glue, liquid dye, or indeed any liquid with appropriate viscosity. In particular, a dispenser device can be used to deliver a measured quantity of a preparation, such as a medicament, into a drink or food that will be consumed by the user. In some instances, it is desirable to mix a preparation (which may include one or more Cannabis constituents) into a glass of water, coffee, soda, or the like before consumption. In some cases, a preparation may benefit from being combined with another fluid such as a beverage, because the preparation (e.g. containing an oil-based vitamin) may have a bad or sour taste if consumed directly. Exemplary dispenser devices and methods disclosed herein can be used to accurately measure and dispense lubricating oil onto a mechanical mechanism to aid in its smooth function and preservation. In many such applications of lubricating oil it may be imperative that the oil not migrate over time beyond the mechanism, thus the need to dispense only by precise means.

According to some embodiments of the present invention, a dispenser device can be used to deliver one or more doses of a preparation (e.g. containing one or more medicamental or Cannabis oils) into a drink, whereby the user can mask an unpleasant or undesirable taste of the medicament. In some cases, a dispenser device can be used for the dosing of medicamental or Cannabis oils. In some cases, a dispenser device can be used to deliver any desired formulary of a medicament or preparation, in order to provide maximum benefit or desired effect to the user. Dispenser devices can be used to deliver precisely measured doses, in a repeatable and reliable manner, with little need for the user to manipulate or measure the medicament itself. In some cases, a dispenser device does not include a needle, an internal or external spike, or a barb.

In some cases, a dispenser device is therefore safer than a syringe. In some cases, a dispenser device is portable, socially acceptable when carried in the open pocket of a shirt, protected from accidental disassembly, and easily adjustable to be set to deliver one of several preset dose amounts without fuss by the user. Exemplary dispenser devices can be used to deliver repeatable and precise dosing of medicaments or preparations upon demand. Embodiments of the present invention encompass dispenser devices that do not leak liquids (e.g. from a vial and/or a dispenser tip), even when a cap is not present on the dispenser device. Exemplary dispenser devices do not require the replacement in installation of a needle or a similar consumable component, prior to the dispensing of a preparation or liquid.

According to some embodiments, a dispenser device can be provided as a small portable device that delivers small pre-measured amounts of a preparation or liquid, not for injection, using a method that is accurate and repeatable, and does not employ an internal or external spike, barb, or needle (e.g. tissue-penetrating features, such as a standard tissue penetrating hypodermic needle) that can injure a user.

Embodiments of the present invention encompass dispenser devices and methods for use in effecting the controlled and measured dispensing of preparations or liquids, such as a dose of a medicament or preparation to food or drink. In some cases, a dispenser device can be used to dispense oils. In some cases, a dispenser device can have the appearance and form-factor of a pen, perhaps similar in size and shape of a high-quality fountain pen. In some cases, dispenser devices can include disposable cartridges or vials that can hold medicaments of differing formulations. In some cases, dispenser devices can employ cartridges or vials without the need of a foil or paper seals that must be pierced by a pin or needle before prior to, or upon or during, the installation of the cartridge into the device.

According to some embodiments, a vial, cartridge, and/or dispenser device can be configured so that a partially used cartridge or vial may be removed and stored safely and cleanly, while another cartridge or vial is installed and used. In some cases, a vial or cartridge may operate to contain the contents therein (e.g. an amount of a preparation) without requiring a seal.

According to some embodiments, the term “fixed amount” as used herein may refer to an amount of a preparation, which may be for example a lubricating oil, a paint, a glue, a catalyst, another chemical, a dose of medicament or one or more Cannabis constituents.

In some cases, a dispensing device can be configured to be controlled by or in communication with a software application or other programming mechanism, whereby the software application or programming mechanism can convert mg (milligrams) of one or more Cannabis constituents or preparations into a corresponding setting for a control assembly, and whereby data can be collected for verifiable analytics. According to some embodiments, a dispensing device can be provided as a single chamber micro-dosing device configured to delivery whole plant cannabis oil and tinctures for oral ingestion. In some cases, a dispenser device can be configured for Bluetooth connectivity, and can be configured to be controlled by or in communication with a controlling dose tracking application.

According to some embodiments, for example in the instance of an electronic version of a dispensing device, a radio-link or infra-red link to a smartphone or other electronic device could be used to set the fixed-amount of preparation for dispensing. In some cases, a control assembly may be of an electronic design in part or in full, and a dispense button or fixed-amount activation button may omitted in favor of using a radio-link or infra-red link to a smartphone or other electronic device for activation and delivery of the fixed-amount. In some cases, a dispenser device can be configured to be controlled by or in communication with a software application or other programming mechanism, whereby data can be collected for verifiable analytics.

Cannabis and Other Preparations

As discussed elsewhere herein, a preparation (e.g. preparation 344 as depicted in FIG. 3) may be provided as a fluid. In some cases, preparation 344 may include a medicament. In some cases, preparation 344 may include a Cannabis oil, tincture, extract, and/or derivative. A dispensing device may be configured to dispense any of a variety of preparations, such as fluids, gels, pastes, liquids, and other flowable materials. In some cases, a preparation 344 may include a whole plant Cannabis oil. In some cases, a preparation 344 may include a whole plant Cannabis tincture.

In exemplary embodiments, preparation 344 may include one or more substances derived from a Cannabis plant (e.g. Cannabis indica, Cannabis sativa, or Cannabis ruderalis). Derived substances may include cannabinoids, terpenes, and flavonoids. According to some embodiments, these, and other derivatives or compounds obtained from a Cannabis plant, may be referred to generically as Cannabis constituents.

Cannabinoids are a unique class of compounds associated with the Cannabis plant. According to some embodiments, Cannabis cannabinoids may include cannabichromenes such as cannabichromene (CBC), cannabichromenic acid (CBCA), cannabichromevarin (CBCV), and cannabichromevarinic acid (CBCVA). According to some embodiments, cannabinoids may include cannabicyclols such as cannabicyclol (CBL), cannabicyclolic acid (CBLA), and cannabicyclovarin (CBLV). According to some embodiments, cannabinoids may include cannabidiols such as cannabidiol (CBD), cannabidiol monomethylether (CBDM), cannabidiolic acid (CBDA), cannabidiorcol (CBD-C1), cannabidivarin (CBDV), and cannabidivarinic acid (CBDVA). According to some embodiments, cannabinoids may include cannabielsoins such as cannabielsoic acid B (CBEA-B), cannabielsoin (CBE), and cannabielsoin acid A (CBEA-A). According to some embodiments, cannabinoids may include cannabigerols such as cannabigerol (CBG), cannabigerol monomethylether (CBGM), cannabigerolic acid (CBGA), cannabigerolic acid monomethylether (CBGAM), cannabigerovarin (CBGV), and cannabigerovarinic acid (CBGVA). According to some embodiments, cannabinoids may include cannabinols and cannabinodiols such as cannabinodiol (CBND), cannabinodivarin (CBVD), cannabinol (CBN), cannabinol methylether (CBNM), cannabinol-C2 (CBN-C2), cannabinol-C4 (CBN-C4), cannabinolic acid (CBNA), cannabiorcool (CBN-C1), and cannabivarin (CBV). According to some embodiments, cannabinoids may include cannabitriols such as 10-Ethoxy-9-hydroxy-delta-6a-tetrahydrocannabinol, 8,9-Dihydroxy-delta-6a-tetrahydrocannabinol, cannabitriol (CBT), and cannabitriolvarin (CBTV). According to some embodiments, cannabinoids may include delta-8-tetrahydrocannabinols such as delta-8-tetrahydrocannabinol (Δ8-THC), and delta-8-tetrahydrocannabinolic acid (Δ8-THCA). According to some embodiments, cannabinoids may include delta-9-tetrahydrocannabinols such as delta-9-tetrahydrocannabinol (THC), delta-9-tetrahydrocannabinol-C4 (THC-C4), delta-9-tetrahydrocannabinolic acid A (THCA-A), delta-9-tetrahydrocannabinolic acid B (THCA-B), delta-9-tetrahydrocannabinolic acid-C4 (THCA-C4), delta-9-tetrahydrocannabiorcol (THC-C1), delta-9-tetrahydrocannabiorcolic acid (THCA-C1), delta-9-tetrahydrocannabivarin (THCV), and delta-9-tetrahydrocannabivarinic acid (THCVA). According to some embodiments, cannabinoids may include other cannabinoids such as 10-Oxo-delta-6a-tetrahydrocannabinol (OTHC), cannabichromanon (CBCF), cannabifuran (CBF), cannabiglendol, cannabiripsol (CBR), cannabicitran (CBT), dehydrocannabifuran (DCBF), delta-9-cis-tetrahydrocannabinol (cis-THC), tryhydroxy-delta-9-tetrahydrocannabinol (triOH-THC), and 3,4,5,6-Tetrahydro-7-hydroxy-alpha-alpha-2-trimethyl-9-n-propyl-2,6-methano-2H-1-benzoxocin-5-methanol (OH-iso-HHCV). In some cases, certain cannabinoids obtained from raw or live Cannabis can be converted by a decarboxylation process to provide different cannabinoids. For example, tetrahydrocannabinolic acid (THCA) obtained from raw or live Cannabis can be converted by a decarboxylation process to provide tetrahydrocannabinol (THC). Similarly, cannabidiolic acid (CBDA) obtained from raw or live Cannabis can be converted by a decarboxylation process to provide cannabidiol (CBD).

Terpenes are aromatic compounds associated with the Cannabis plant. According to some embodiments, Cannabis terpenes may include myrcene, linalool, pinene, limonene, β-caryophyllene, α-humulene, ocimene, terpinolene, geraniol, terpineol, valencene, phellandrene, carene, fenchol, terpinene, borneol, bisabolol, camphene, phytol, camphor, sabinene, isoborneol, menthol, nerolidol, cedrane, isopulegol, cymene, pulegone, eucalyptol, geranyl acetate, and guaiol.

Flavonoids are secondary metabolites associated with the Cannabis plant. According to some embodiments, Cannabis flavonoids may include cannflavine A, cannflavine B, cannflavine C, vitexin, isovitexin, apigenin, kaempferol, quercetin, luteolin, and orientin.

THC is probably best known for being the main psychoactive ingredient in Cannabis. CBD, however, is generally considered to be non-psychoactive. In some cases, it may be desirable to provide a user or consumer with a particular combination of Cannabis constituents within a preparation. For example, it may be desirable to provide a user or consumer with a preparation having a CBD:THC ratio of 1:1. In other instances, it may be desirable to provide a user or consumer with a preparation having a CBD:THC ratio of 10:1. Embodiments of the present invention encompass preparations having any desired ratio and/or combination of Cannabis constituents.

Consumers, patients, and other individuals may prefer and/or require individual Cannabis constituents (e.g. cannabinoid, terpene, and/or flavonoid compounds) to be delivered in a specific relationship with each other in order to administer a desired dose.

In some cases, a preparation may include an active ingredient, such as a botanical active ingredient. In some cases, a preparation may include an inactive ingredient. In some cases, a preparation may include a cannabinoid. In some cases, a preparation may include a constituent derived from a botanical such as a plant other botanical organism. In some cases, a water soluble liquid preparation can include one or more cannabinoid oils or terpenes. In some cases, a preparation may include one or more of: cetirizine, ibuprofen, naproxen, omeprazole, doxylamine, diphenhydramine, melatonin, or meclizine. In some cases, a preparation may include one or more of: a polyphonel, short chain fatty acids, medium chain fatty acids, long chain fatty acids, a green tea catechin, caffeine, a phenol, a glycoside, a labdane diterpenoid, yohimbine, a proanthocyanidin, terpene glycoside, an omega fatty acid, echinacoside, an alkaloid, isovaleric acid, a terpene, gamma-aminobutyric acid, a senna glycoside, cinnamaldehyde, or any vitamin such as Vitamin D.

According to some embodiments, a preparation may include a Cannabis infusion, such as an extract obtained by soaking the leaves or other parts of a Cannabis plant in a liquid. In some cases, a preparation may include a water soluble liquid compound that is a cannabinoid oil or a terpene. Typically, cannabinoids (e.g. CBD, THC, and the like) are hydrophobic substances. Cannabinoids can, however, be formulated to be water-compatible and appear water-soluble. For example, CBD, THC, and other oils can be made water-compatible if they are formulated as oil-in-water nanoemulsions, which are stable and visually homogeneous oil/water mixtures. Nanoemulsions can be prepared in concentrated forms (e.g. tens of mg/ml) that are fully miscible with water and, therefore, appear water-soluble. In addition, nanoemulsions can be made translucent and practically tasteless, which means that they can be mixed into water without compromising its optical clarity or taste. According to some embodiments, nanoemulsions can be beneficial because they can be easily mixed into beverages, translucent, low-taste, and safe for consumption. Nanoemulsions can also provide exceptionally high bioavailability, fast onset of action, stable blood levels and high CBD/THC loading capacities. Embodiments of the present invention encompass mechanical dispensing devices used in combination with nanoemulsion technology to provide a precise, high water soluble, and bioavailable product for the oral consumption of constituents such as Cannabis oils.

As discussed elsewhere herein, a preparation (e.g. preparation 344 as depicted in FIG. 3) may be provided as a fluid. In some cases, preparation 344 may include a medicament. In some cases, preparation 344 may include one or more substances derived from a plant or fungi. In some cases, preparation 344 may include one or more substances derived from a Psilocybe fungi (e.g. Psilocybe cubensis, Psilocybe azurescens, Psilocybe caerulescens, Psilocybe mexicana, Psilocybe quebecensis, Psilocybe baeocystis, Psilocybe cyanescens, Psilocybe semilanceata, and the like). Derived substances may include Psilocybin (4-phosphoryloxy-N,N-dimethyltryptamine), Psilocin (4-hydroxy-N,N-dimethyltryptamine), Baeocystin (4-phosphoryloxy-N-methyltryptamine), Norbaeocystin (4-phosphoryloxytryptamine), Norpsilocin, Aerugunosin, and the like. According to some embodiments, these, and other derivatives or compounds obtained from a Psilocybe organism, may be referred to generically as Psilocybe constituents. Likewise, a preparation 344 may include one or more substances or constituents derived from other psilocybin containing mushrooms, including fungi from the genera of Copelandia, Gymnopilus, Inocybe, Panaeolus, Pholiotina, and Pluteus. In some cases, a preparation 344 may include lysergic acid diethylamide (LSD). In some cases, a preparation 344 may include 3,4-methylenedioxymethamphetamine (MDMA). In some cases, a preparation 344 may include ketamine. In some cases, a preparation 344 may include one or more psychoactive alkaloids, including indole alkaloids. In some cases, preparation 344 may include one or more substances derived from Anadenanthera colubrina or Anadenanthera peregrina. In some cases, a preparation may include any compound, substance, or chemical which is believed to be useful in treating individuals having or susceptible to depression, anxiety, and posttraumatic stress disorder (PTSD). In some cases, a preparation 344 may include one or more materials, chemicals, or compounds selected from the group consisting of a monoamine oxidase inhibitor, dimethyltryptamine, ibogaine, salvinorin A, salvinorin B, 3,4,5-trimethoxyphenethylamine (mescaline), bufotenine, 5-methoxy-N,N-dimethyltryptamine (5-MeO-DMT), mesembrine, mesembrenone, mesembrenol, tortuosamine, muscimol, muscarine, muscazone, ibotenic acid, and 5-bromo-N,N-dimethyltryptamine (5-Bromo-DMT). A monoamine oxidase (MAO) inhibitor may include a β-carboline. A monoamine oxidase (MAO) inhibitor may include a nonselective MAO-A/MAO-B inhibitor, a selective MAO-A inhibitor, a selective MAO-B inhibitor, or a reversible inhibitors of monoamine oxidase A (RIMA). RIMAs are a subclass of MAO inhibitors that selectively and reversibly inhibit the MAO-A enzyme. Exemplary nonselective MAO-A/MAO-B inhibitors include hydrazines such as isocarboxazid, nialamide, phenelzine, and hydracarbazine, as well as non-hydrazines such as tranylcypromine. Selective MAO-A inhibitors include bifemelane, moclobemide, pirlindole, and toloxatone. Selective MAO-B inhibitors include rasagiline, selegiline, and safinamide. Reversible inhibitors of monoamine oxidase A include brofaromine, caroxazone, eprobemide, Methylene blue, metralindole, minaprine, moclobemide, pirlindole, toloxatone, curcumin, harmaline, and harmine. In some cases, a preparation 344 may include one or more materials, chemicals, or compounds described in any one or more of U.S. Pat. No. 3,075,992 (Hofmann), U.S. Pat. No. 3,183,172 (Heim et al.), U.S. Pat. No. 3,192,111 (Hofmann et al.), and/or US Patent Publication Nos. 2018/0021326 (Stamets), 2018/0221396 (Chadeayne), 2019/0105313 (Stamets), 2019/0119310 (Londesbrough et al.), 2019/0142851 (Chadeayne), and/or 2019/0350949 (Kucuksen et al.). The content of each of the above patent filings is incorporated herein by reference.

Micro-Dosing

According to some embodiments, it may be desirable to precisely control the amount of one or more Cannabis constituents, or other compounds or molecules, that are being dispensed from a dispensing device. Embodiments of the present invention enable the user or individual to precisely vary or control the quantity of cannabinoids (or other compounds or molecules) that are dispensed from a dispensing device. For example, the quantity of a preparation or fluid that is dispensed from a dispensing device may be varied by changing the dose settings of the dispensing device. The user or healthcare professional may then select the most appropriate micro-dosing quantity for a particular treatment regime. The ability to control the dose of one cannabinoid (such as THC) to a custom level or mixture of cannabinoids (such as CBD) may give the healthcare professionals and users the flexibility to titrate the Cannabis constituents and/or other compounds or molecules upward or downward with more precision. In this way, for example through an iterative process, the user or individual is able to discover a minimum amount or dosage of a Cannabis preparation that provides the desired result.

According to some embodiments, the micro-dosing of Cannabis constituents involves taking a very measured micro-dose of THC, which is typically considered to be a psychoactive ingredient in Cannabis, and a measured dose of CBD, which is typically considered to be a non-psychoactive ingredient in Cannabis. Toward this end, vials containing Cannabis preparations can be provided which contain known amounts, ratios, and/or concentrations of Cannabis constituents. For example, a vial may contain 3 ml of a preparation that contains THC at a concentration of 25 mg/ml and CBD at a concentration of 100 mg/ml. When a dispensing device is used to dispense 0.20 ml (milliliter) of the preparation (e.g. 20 clicks), the user or individual will know that the dispensed amount contains 5 mg of THC and 20 mg of CBD. In some cases, vials or vial assemblies can be provided as pre-mixed or pre-prepared vial assemblies. According to some embodiments, vials can be prepared so as to contain one or more Cannabis constituents, in any desired combination, in any desired amount or ratio of amounts.

Because precise micro-dose amounts can be accurately dispensed using the dispensing devices and methods as disclosed herein, it is possible for users or individuals to obtain the desired dosage in an economical fashion while also minimizing waste and avoiding inadvertent or unwanted overdosing of the Cannabis constituents and/or other compounds or molecules.

Embodiments of the present invention are well suited for use in delivering a dose or an amount of a preparation for consumption by an individual or user. Any aspect of the preparation, including for example the concentration or amount of one or more Cannabis constituents contained therein, can be designed as desire. In some cases, the formulation and/or amount of the preparation to be consumed is based on a therapeutic relationship amongst one or more components of the preparation. In some case, a therapeutic relationship can be determined between individual cannabinoid compounds, and a dispenser device can help ensure that a patient/user receives an optimum therapeutic dose without the inherent risks associated with bottle droppers, smoking, vaping, or edibles. In some cases, a preparation may include one or more cannabinoids and/or terpenes present in water soluble fluids that make it easy to dispense, dissolve, and mix into beverage of choice.

Table 1 below illustrates the operability of exemplary dispensing device configurations, according to embodiments of the present invention. According to some embodiments, the total milligrams in this example vial can be 300. According to some embodiments, this example can be based on 1 milligram of active cannabinoid per units dispensed or displayed on screen.

TABLE 1

| Vial ml | THC mg | Keypad Button Units |
|---|---|---|
| 3 | 30 | 30 |
| 1 | 10 | 10 |
| 0.1 | 1 | 1 |
| 0.05 | 0.05 | 0.5 |

In some cases, a dispensing device can include a dosage selection mechanism, such as an interface or keypad having UP/DOWN buttons, to assist the user in controlling how much preparation is dispensed with each device actuation (e.g. pressing GO button). In some cases, a dispensing device is configured to dispense 1/100 of milliliter of preparation per actuation. In some cases, a dispenser device is configured to dispense less than a drop (e.g. a droplet) of preparation per actuation. In some cases, a dispenser device is configured to dispense the contents of a vial upon actuation of 100 actuations. In some cases, a dosage amount of 2.5 mg or less of Cannabis constituent is considered to be a micro-dose. In some cases, a dosage amount on the order of micrograms is considered to be an ultra-micro-dose.

In some cases, a dispenser device can include or be used with a 3 ml vial filled with a Cannabis extraction oil formulated for oral and sublingual dosing. In some cases, a vial may include 90 mg of active cannabinoids, optionally infused with terpenes and/or nutraceuticals, to provide an entourage effect. Micro-dosing embodiments disclosed herein may be used by individuals having various conditions, including depression, stress, anxiety, pain, and the like. Micro-dosing embodiments disclosed herein may be used by individuals seeking improved mental focus and/or sleeping patterns. As described elsewhere herein, a vial may contain 3 ml of a preparation that contains cannabinoid content at a concentration of 25 mg/ml or 100 mg/ml, for example. In some cases, a vial may contain 3 ml of oil and 100 mg THC. In some cases, a vial may contain a cannabinoid concentration of about 100 mg per 3 ml of oil or other carrier. In some cases, a vial may contain a cannabinoid concentration of about 100 mg per ml of oil or other carrier. In some cases, a vial may contain a cannabinoid concentration having a value within a range from about 0.3 mg per ml of carrier to about 500 mg per ml of carrier. In some cases, a vial may include cannabinoid content having within a range of about 1 mg to about 1500 mg. In some cases, the cannabinoid content includes active cannabinoids. In some cases, the cannabinoid content contained within a vial may have a value of about 1.5 ml, or less. In some cases, a 3 ml vial may include 1.5 ml of cannabinoids, and 1.5 ml of carrier or blending oil, which may be olive oil or medium-chain triglyceride (MCT) oil.

Mobile Applications, Computerized Delivery Devices, Graphic User Interfaces

Embodiments of the present invention encompass mobile applications for use with chambered dispensing devices, as well as graphic user interfaces for such mobile applications or chambered dispensing devices. A mobile application can allow an individual or patient to interact with a chambered dispensing device via a mobile computing device, such as a smart phone. A mobile application can run on a handheld device (e.g. smart phone), and in some instances, the handheld device can connect with a remote server containing data, and that stores data and/or processes data for use on the handheld device. In some cases, embodiments of the present invention encompass a graphic user interface for a display screen for a handheld device or chambered dispensing device. In some cases, embodiments of the present invention encompass a display screen with a graphical user interface for handheld device that can be used to control a chambered dispensing device. In some cases, embodiments of the present invention encompass a display screen with a graphical user interface for a chambered dispensing device. In some cases, a handheld device, which may also be referred to as a mobile computing device, may include a display screen and a processor, and can be configured to display various graphical user interface elements as disclosed herein.

An exemplary mobile computing device can include a housing configured to internally house various electronic components such as a processor, volatile storage device, non-volatile storage device, a display, an input subsystem, and the like. In some cases, a mobile computing device can include a processor having one or more physical devices configured to execute instructions. For example, the processor may be configured to execute instructions that are part of one or more applications, programs, routines, libraries, objects, components, data structures, or other logical constructs. Such instructions may be implemented to perform a task, implement a data type, transform the state of one or more components, achieve a technical effect, or otherwise arrive at a desired result, for example any of the results disclosed herein. A processor may include one or more physical processors (hardware) configured to execute software instructions. Additionally or alternatively, the processor may include one or more hardware logic circuits or firmware devices configured to execute hardware-implemented logic or firmware instructions. A mobile computing device can include a communication subsystem that is configured to communicatively couple various computing devices described herein with each other, and with other devices. A communication subsystem may include wired and/or wireless communication devices compatible with one or more different communication protocols. As non-limiting examples, a communication subsystem may be configured for communication via a wireless telephone network, or a wired or wireless local- or wide-area network, such as a HDMI over Wi-Fi connection. In some embodiments, a communication subsystem may allow a mobile computing device to send and/or receive messages to and/or from other devices via a network such as the Internet.

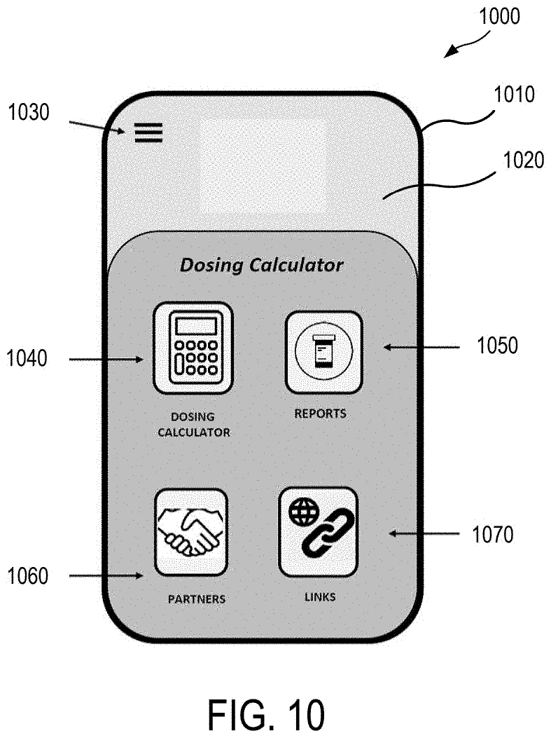

FIG. 10 depicts aspects of a mobile computing device 1000 having a display screen 1010 with a graphical user interface 1020. As shown here, user interface 1020 includes a menu option 1030 that opens to “About”, “My Account”, “Settings”, and “Disclaimer” options. The “About” option can open to information regarding the device manufacturer or designer, the software version, a feedback function, a terms of service and privacy policy function, and social media icons. The “My Account” option can open to information regarding the profile, settings (e.g. scheduling, reminders, security and privacy, history). The user interface 1020 also includes a dosing calculator option 1040 that can open to a dosing calculator page. Further, the user interface 1020 can include a reports option 1050 that can open to a reports page. According to some embodiments, selecting the reports icon 1050 can open a feedback report chart which shows the number of doses and mg/dose over a period of time selected by the user (e.g. week, month, 3 months, 6 months, year). Using the reports option 1050, it is possible to download the dosing history (e.g. to a spreadsheet such as an Excel document) and that will automatically send to their user email address provided in the profile. The user interface 1020 also includes a partners option 1060 that can open to a page containing links to partner websites, which may be listed on a manufacturer or designer website. The user interface 1020 also includes a links option 1070 that can open to a page containing links dosing guidelines published by peer reviewed and medical doctors (e.g. informational only), manufacturer or designer partners, and/or a “find us” or “where to buy” page.

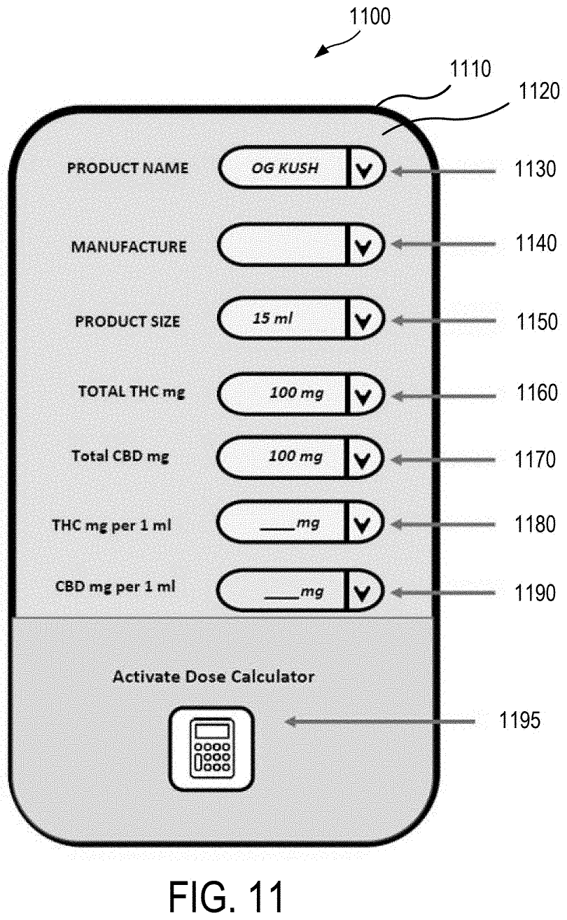

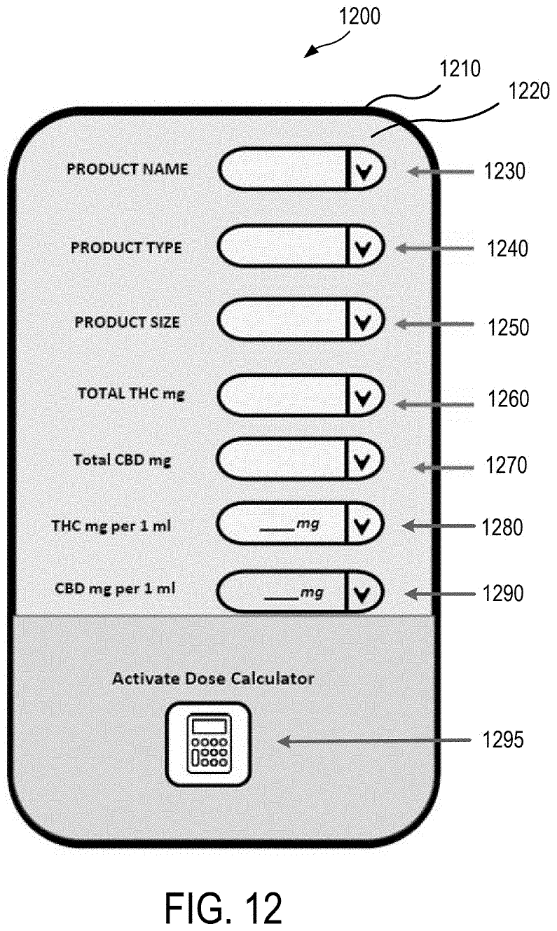

FIG. 11 depicts aspects of a mobile computing device 1100 having a display screen 1110 with a graphical user interface 1120. The page shown here may be referred to as a dosing calculator page. As illustrated here, user interface 1120 includes a product name function 1130 that allows the selection or designation of the name of a strain, the name of a tincture formula, the name of a custom oil extract, or the like. User interface 1120 includes a manufacture function 1140 that allows the selection or designation of the name of a brand or manufacturer. An “other” option can be selected, which can link to the screen depicted in FIG. 12. Graphical user interface 1120 can also include a product size function 1150 that allows the selection or designation of a product size (e.g. vial or chamber), such as 15 ml or 30 ml. Graphical user interface 1120 can include a total THC function 1160 that allows the selection or designation of the total number of milligrams of THC (e.g. as provided on a product label). Graphical user interface 1120 can include a total CBD function 1170 that allows the selection or designation of the total number of milligrams of CBD (e.g. as provided on a product label). Graphical user interface 1120 can include a THC mg per 1 ml function 1180 that provides the result of a calculation based on the formula total mg/number of ml. Graphical user interface 1120 can include a THC mg per 1 ml function 1190 that provides the result of a calculation based on the formula total mg/number of ml. Graphical user interface 1120 can include an activate dose calculator function 1195 that, when this option is selected, displays a dose calculator pop-up having a keypad that allows the user to enter ratio information.

FIG. 12 depicts aspects of a mobile computing device 1200 having a display screen 1210 with a graphical user interface 1220. The page illustrated here may be displayed when “other” is selected via user input in the product name option 1230 shown in FIG. 11. As illustrated here, user interface 1220 includes a product name function 1230 that allows the selection or designation of the name of a Cannabis strain name, the name of a tincture formula, the name of a custom oil extract, or the like. This information can be entered by the user. User interface 1220 includes a product type function 1240 that allows the selection or designation of the name of a brand or manufacturer, or the selection or designation of the product type. Graphical user interface 1220 can also include a product size function 1250 that allows the selection or designation of a product size (e.g. vial or chamber), such as 15 ml, 30 ml, or any other desired volume. Graphical user interface 1220 can include a total THC function 1260 that allows the selection or designation of the total number of milligrams of THC (e.g. as provided on a product label). Graphical user interface 1220 can include a total CBD function 1270 that allows the selection or designation of the total number of milligrams of CBD (e.g. as provided on a product label). Graphical user interface 1220 can include a THC mg per 1 ml function 1280 that provides the result of a calculation based on the formula total mg/number of ml. Graphical user interface 1220 can include a THC mg per 1 ml function 1290 that provides the result of a calculation based on the formula total mg/number of ml. Graphical user interface 1220 can include an activate dose calculator function 1295 that, when this option is selected, displays a dose calculator pop-up having a keypad that allows the user to enter ratio information.

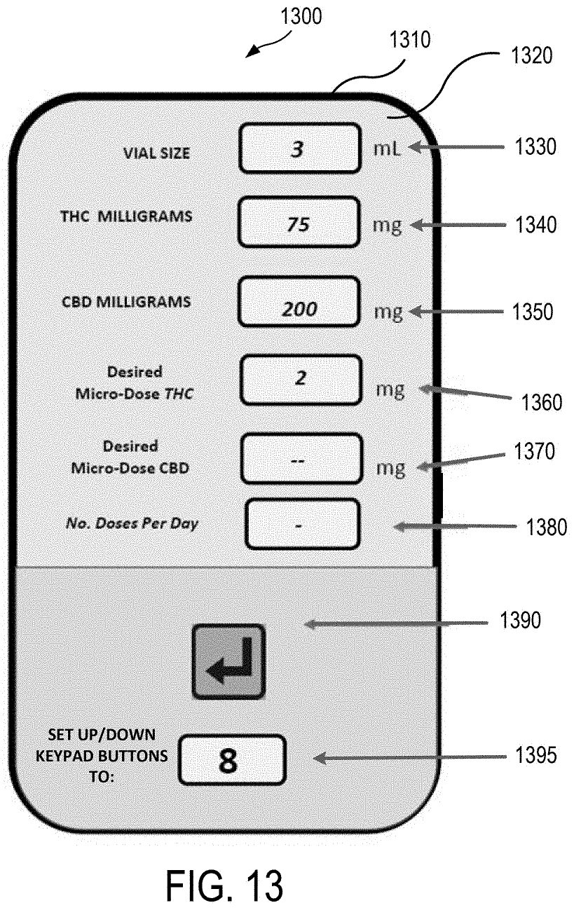

FIG. 13 depicts aspects of a mobile computing device 1300 having a display screen 1310 with a graphical user interface 1320. The page shown here may be referred to as an activated dose calculator page. As illustrated here, user interface 1320 includes a vial size function 1330 that allows the selection or designation of a vial size. User interface 1320 also includes a THC milligrams function 1340 that allows the selection or designation of a milligrams of THC value and a CBD milligrams function 1350 that allows the selection or designation of a milligrams of CBD value. Further, user interface 1320 includes a desired micro-dose THC function 1360 that allows the selection or designation of a desired micro-dose of THC, in milligrams for example. User interface 1320 also includes a desired micro-dose CBD function 1370 that allows the selection or designation of a desired micro-dose of CBD, in milligrams for example. According to some embodiments, the user interface 1320 is configured so that a user or individual can select either THC or CBD for micro-dosing, and the user cannot select both THC and CBD at the same time. User interface 1320 also includes a notification function 1380 to provide a dosing alert (e.g. to provide a user or individual of the number of doses dispensed per day, so as to assist the user or individual in adhering to a desired or prescribed dispensing regimen). According to some embodiments, the focus of the calculator page provided by user interface 1320 is to calculate either a THC dose or a CBD dose, but not both. For the cannabinoid dose which is not calculated, an adjustment can be made based on the THC:CBD ratio. If a recommended THC dose, for example from FIG. 11, is 2 mg, the calculator can provide an indication 1395 to the user or individual of how to configure a dispensing device (e.g. using UP/DOWN keypad buttons) so as to achieve the recommended 2 mg dose. In some embodiments, the calculator can be configured to round to the nearest quarter mg based on the desired dose entered.

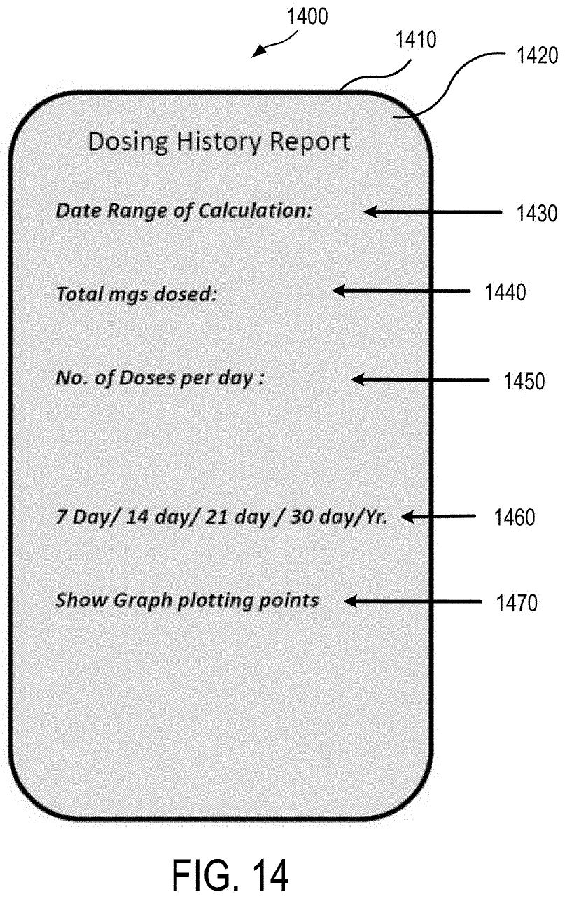

FIG. 14 depicts aspects of a mobile computing device 1400 having a display screen 1410 with a graphical user interface 1420. The page shown here may be referred to as a dosing history report page. As illustrated here, user interface 1420 includes a date range of calculation field 1430, a total amount (e.g. mgs) dosed field 1440, a number of doses per day field 1450, a day or time duration count field 1460, and a show graph plotting points function 1470.

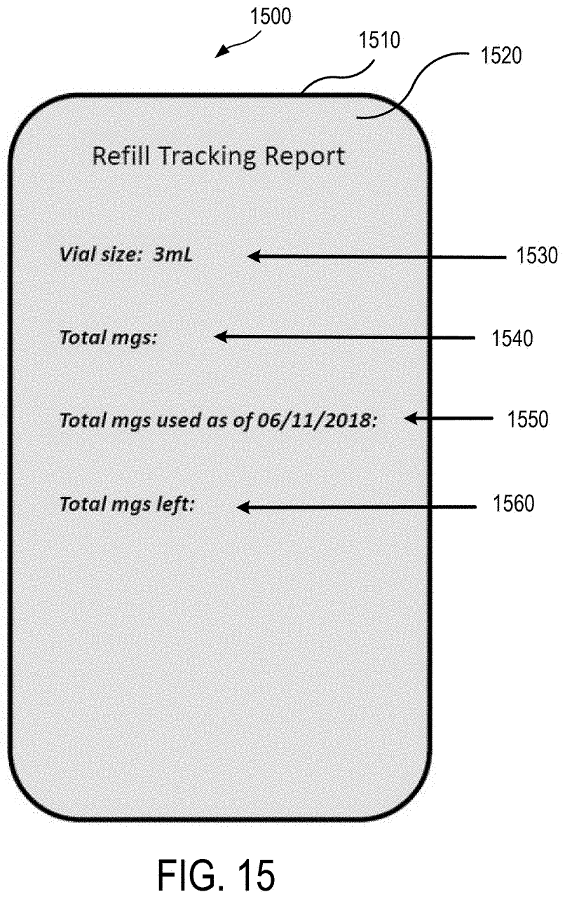

FIG. 15 depicts aspects of a mobile computing device 1500 having a display screen 1510 with a graphical user interface 1520. The page shown here may be referred to as a refill tracking report page. As illustrated here, user interface 1520 includes a vial size field 1530, a total amount (e.g. mgs) field 1540, a total amount (e.g. mgs) used as of a date field 1550, and a total amount (e.g. mgs) remaining field 1560. In addition to displaying a dosing history report page and a refill tracking report page, a graphical user interface may also display a therapeutic history report page.

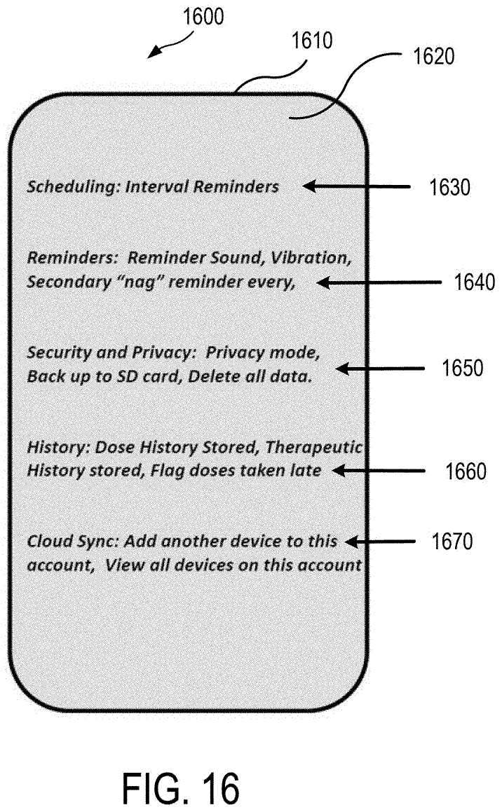

FIG. 16 depicts aspects of a mobile computing device 1600 having a display screen 1610 with a graphical user interface 1620. The page shown here may be referred to as a privacy settings page. As illustrated here, user interface 1620 includes a scheduling field 1630, a reminders field 1640, a security and privacy field 1650, a history field 1660, and a cloud sync field 1670. The scheduling field 1630 enables a user or individual to designate or select interval reminders. The reminders field 1640 enables a user or individual to designate or select a reminder sound, a vibration, and a secondary nag reminder at a particular interval. The security and privacy field 1650 enables a user or individual to designate or select a privacy mode, a back up to SD card operation, and a delete all data operation. The history field 1660 enables a user or individual to access or display history stored data, therapeutic history stored data, and to flag doses taken late. The cloud sync field 1670 enables a user or individual to add another device to an account, and to view all devices on an account.

According to some embodiments, a mobile computing device application can be provided for users or individuals in the wellness/medical Cannabis space. The application can be configured to work in conjunction with single cartridge, dual cartridge, and other multi-cartridge preparation or oil dispensing devices as disclosed herein. In order for users, patients, and other individuals to know how much of a particular preparation or oil to dispense or dose, the application can include a dosing calculator function (e.g. selectable icon) that determines how many numbered units of the dispensing device correspond to a particular milligram dose of the active ingredients in the preparation or oil and records the date, time, and UP/DOWN keypad number of that calculation. A feedback reports function (e.g. selectable icon) can provide the user or individual with a graph that shows the user calculation history according to a selected time frame (e.g. week, month, 3 months, 6 months, year) to display patterns of use history.

In some embodiments, a mobile computing device application can include a security feature that can be used in tightly controlled clinical environments where a patient's adherence and dosing protocol is important. Fingerprint authentication and fingerprint authentication applications that are connected to a computing cloud can authenticate the user/patient opening the mobile computing device application and unlocking the dispensing device.

In some embodiments, a mobile computing device application can include an easy-to-use, passwordless biometric authentication to prevent other users or patients from using the dispensing device and can enable users, caregivers, or nurses to securely register, log in or issue commands to internet of things (IoT) devices via a smartphone application and biometric fingerprint scan.

In some cases, a dispensing device can include a dosage selection mechanism, such as an interface or keypad that can be used to select a number of units for dispensing or dosing. In some cases, a dispensing device can be provided as a single motor digital pen. In a CBD tracking scenario, the dominant cannabinoid present in a preparation or tincture (e.g. in a vial purchased by user or individual) is CBD with very little or no THC. Exemplary dominant CBD tinctures have 20:1 to 30:1 CBD to THC ratios. A user or individual can identify and enter the size of the bottle or vial/chamber (e.g. 15 ml or 30 ml), for example by inputting this information into the dispensing device, and read the label to determine how many milligrams of CBD are total in the bottle. The application can determine the milligrams of CBD per 3 mL vial. Once the application determines milligrams per 3 mL vial, the application can provide a query asking what the desired milligrams per dose the user wants to take or dispense. The user can input the milligrams per dose (e.g. 20 milligrams), then the application can calculate how many units the dispensing device needs to be set or adjusted to. For example, using the arrow buttons up or down, a user can adjust, dial in, or otherwise designate the recommended or desired number of units to dispense 20 milligrams of CBD.

In a THC tracking scenario, the dominant cannabinoid in the preparation or tincture is THC with very little or no CBD. Exemplary dominant THC tinctures have 10:1 to 2:1 THC to CBD ratios. An individual or user can identify and enter the size of the bottle or chamber (e.g. 15 ml or 30 ml) and read the label to determine how many milligrams of THC are total in the bottle. The mobile application can determine the milligrams of THC per 3 mL vial. Once the mobile application determines milligrams per 3 mL vial, the application can query the user or individual, asking what the desired milligrams per dose the user wants to take or dispense. The user or individual can input the milligrams per dose (e.g. 10 milligrams), and the application can calculate how many units the device needs to be dialed to or adjusted. For example, using the arrow buttons up or down, the user can dial in or otherwise select the desired or recommended number of units to dispense 10 milligrams of THC. In some cases, preparations may contain one or more terpenes, which are dispensed along with the dispensed THC and/or CBD.

In some cases, a dispensing device can be provided as a dual motor digital pen. Individual cannabinoid formulas or preparations (e.g. one for THC and one for CBD) can be provided in their own vial, and the respective two vials or chambers can be present or loaded in a dispensing device. In operation, the user or individual can enter the total number of milligrams of THC and CBD that are in each vial (these can be equal in number of milligrams per vial). The mobile application can query the user or individual to ask how many milligrams per dose and at what ratio of THC to CBD the user wants. The user can enter a certain or desired ratio (e.g. 1:1 for THC:CBD) and the application can automatically dial or select the units based on the desired ratio. The application can be configured to query the user or individual to ask for confirmation to dispense, and once confirmed the device can dispense a 1:1 ratio of THC and CBD. In some embodiments, a dispensing device can be configured so that a user or individual can override the ratio dispensing feature of the mobile application and simply use the up/down arrow keys for each vial to dial in their own desired custom ratio.

In some embodiments, the dosage setting can be powered or selected through the mobile application under a dose calculator icon and the patient or user can choose the desired dose of one cannabinoid (e.g. THC, psychoactive) first, and if no such cannabinoid (e.g. THC) is present in the preparation or formulation, then the user or individual can select another desired dose (e.g. another cannabinoid, such as CBD) via the mobile application. In some embodiments, a dispensing device can be configured so that it will default to a prior setting, and dispensed if the application and/or phone is not within range of the device. In some embodiments, the user can use arrows to adjust dose if the application and/or phone informs the device to go on manual mode.

Table 2 below illustrates the operability of exemplary dispensing device configurations, according to embodiments of the present invention. This table shows how a mobile application can calculate the units per dose for a single motor digital device, where a manufacturer is filling vials or chambers with a preparation (e.g. a generic tincture).

TABLE 2
Application conversion chart for single motor digital dispensing device or pen

| | | Input Total | | Application |
|---|---|---|---|---|
| | | milligrams of | | Calculates |
| Identify | Identify Dominant | dominant | | Number of Units |
| Bottle Size | Cannabinoid Need | cannabinoid | Input Desired | Displayed on |
| 15 mL/30 mL | to Track THC/CBD | from bottle | Dose | Screen |
| 15 mL | THC | 250 milligrams | 5 mg | 30 units |
| 30 mL | CBD | 1000 milligrams | 15 mg | 45 units |

According to some embodiments, the application can calculate the total milligrams divided by the bottle size. The application can also calculate the milligrams per milliliter, times three. The application can also calculate the total milligrams in a 3 mL vial. Further, the application can calculate the total units needed by dividing 300 by total milligrams in a 3 mL vial and multiplying this by the desired dose.

According to some embodiments, a glass vial/cartridge or chamber can be tagged or otherwise labeled with a security feature, such as a RFID mechanism. For example, a custom made plastic RFID tagging system can be affixed to or coupled with the outside of a vial/cartridge or chamber, and an RFID sensor included in the dispensing device (e.g. within the PCB design of a digital pen). The RFID sensor can operate to read and authenticate that the vial being used is a proprietary vial and can be used to authorize or give the “OK” for the pen to operate and dispense. In some embodiments, without this authentication the dispenser will not activate and work. Such a security mechanism can add a further feature to a dispensing device so that “non approved” manufacturers of cartridges or chambers cannot cross sell or introduce their products into a particular dispensing or digital pen device. In some embodiments, such a security feature can prevent unauthorized or “non approved” manufacturers of cartridges or chambers from introducing a “Non Approved” liquid substance into a particular dispensing device. In some embodiments, an RFID tag (e.g. external RFID tag) can be added to or coupled with a vial, and the dispensing device can be configured to use the RFID tag so that the vial can only be used once. In some embodiments, an RFID tag (e.g. external RFID tag) can be added to or coupled with a vial, and the dispensing device can be configured to use the RFID tag so that the dispensing device can only be activated after verification of the vial via the RFID tag is authenticated. In some embodiments, an RFID tagging system can be used for the vials, which may include a passive UHF 860-900 MHz frequency tag made of paper or aluminum foil. This tag can easily adhere to any surface and can allow for the device computer to authenticate the vial and would give permission for dispensing device to operate. The computer main server can track tags that have been used and emptied and restrict future activation of a vial with the same Tag ID number. In some embodiments, an RFID reader can be embedded in the dispensing device, can read tag numbers, and send information back to main server for authentication. In some embodiments, a custom snap-on plastic ring can be coupled with or placed unto the surface of the glass vial. Once the vial is loaded into the device, part of the plastic ring can push and activate a micro switch in the dispenser device which can activate the dispenser device and allow for dispensing.

In some embodiments, a mobile computing device application can include an industry links function or icon, whereby a user or individual can find or access links to dosing guidelines published by peer reviewed and medical doctors (e.g. informational only), device manufacturer or designer partners, and/or a “find us” or “where to buy” page.

In some embodiments, a mobile computing device application can enable a user, patient, or other individual to achieve a precise, controlled, and consistent method for oral consumption of Cannabis whole plant oils or tinctures. This may be particular helpful for individuals who are sensitive to THC, fearful of other oral dosing methods (e.g. edibles), or otherwise apprehensive about consuming more Cannabis that desired. In use, an individual can open the mobile computing device application, and enter their profile information. The application will then take the individual to a home screen that displays various options such as a dose calculator and a reports and links option (e.g. that includes a dosing guidelines link, and/or sites of partners and where to purchase the dispensing devices and/or preparation chambers). The individual can enter information related to the product information and type in the THC to CBD (cannabinoid) ratios and their recommended dose. The calculator can automatically convert those ratios into “units” on the dispensing device, giving the user a precise way to dose the preparation or medicine. Individuals can use the mobile computing device application to track the history of their doses and use that information to make adjustments if needed or desired. According to some embodiments, a mobile computing device application can be configured to enable an administrator to log into a server to download the secured data captured from each user. That data can be used to print reports or graphs, or to make future recommendations on how the application should be used. A data scientist can organize this data in such a way that it is valuable to researchers and product formulators in the industry.

According to some embodiments, a delivery system has a software driven, battery powered computer processor or printed circuit board, coupled to two or more stepper motors with a plunging feature allowing for the software control and tracking of each precise dose.

In some cases, a chambered dispensing device can have one, two, three, or more chambers, with a single delivery system in operative associate with a stepper motor, with a solenoid-driven mechanism that can be used to operate an individual chamber or plunger, or multiple chamber/plunger assemblies separately, so as to control the dose emitted by each chamber, without the expense or weight of having multiple motors.

According to some embodiments, a chambered dispensing device or dispenser unit has an interface to cell phone or tablet devices, such as Wi-Fi or Bluetooth if wireless, USB or Ethernet if wired, whereby a mobile device can operate as a controlling device for a chambered dispensing device. Any of a variety of methods or backbones for interfacing with the controlling device can be used.

Embodiments of the present invention encompass hand held single or multiple chamber or cavity dispensing delivery devices and methods. Exemplary chambered dispensing devices can include a connecting body configured for accepting multiple preparation or liquid compound vials/cartridges for attachment. A connecting body may include a location for one, two, three, or more separate reservoirs or chambers. Exemplary modules, vials, or chambers containing a preparation or liquid compound can be used and configured for fluid communication with a delivery spout. Exemplary chambered dispensing devices can include a microprocessor controlled circuit or computer processor that serves as a controller for the device.

In some cases, a chambered dispensing device can be configured to deliver a preparation or medicament set to a particular amount or dose from a chamber or cartridge, a sensor unit configured to detect a dispensed amount or dose of preparation, for example based on positions and/or movements of the dose setting and dispensing mechanism, and an electronics unit (e.g. having a computer processor) in communication with the sensor unit to process the detected dispensed dose and time data associated with a dispensing event and to wirelessly transmit the dose data to a user's device, such as a smart phone or other mobile computing or communication device. A mobile communication device can include or provide a software application to provide the user with health or use information related to the processed dose data.

According to some embodiments, a sensor unit can be configured to detect a dispensed dose based on one or more positions, settings, and/or movements of a dose setting and dispensing mechanism. An electronics unit may be in communication with a sensor unit, which may include a processing unit including a processor and memory to process the detected dispensed dose and time data associated with a dispensing event to generate dose data, a transmitter to wireless transmit the dose data to a user's device, and a power source to provide electrical power to the electronics unit. A mobile communication device such as a smart phone may include a data processing unit including a processor to process the dose data and a memory to store or buffer the dose data, a display to present a user interface to a user, and a wireless communications unit to wirelessly receive the dose data from a dispensing device.

According to some embodiments, a chambered dispensing system, which may also be referred to as an intelligent preparation or medicinal oil administration, can include a preparation or medicament dispensing device, in communication with a handheld device (e.g. a user's or patient's handheld device, such as a smartphone), in which the dispensing device can be configured to detect and/or record dose sizes that are dispensed. A handheld device can include a software application having a dose calculator that may suggest the dose a user or patient should set on the dispensing device, and can be configured to provide control over several functionalities of a dispensing device. Multiple embodiments of the dispensing device may include various features, including a sensor to detect when the device is being operated, an sensor to detect the dose setting, a sensor to monitor temperature of the preparation or oil, data processing, storage and communication capabilities, and control and messaging (e.g. alert) features to affect the user's or patient's operation of the device.

According to some embodiments, a chambered dispensing device can be operable with a smartphone application which records the time and dosage of the preparation or medicament dispensed. A dispensing device or delivery device can be equipped with a wireless communication module, such as for using a Bluetooth connection. According to some embodiments, a dispensing or delivery device can also have a touchscreen, and can be operated without a smartphone. In some cases, a dispensing system includes a smartphone. According to some embodiments, a user can access the time and dosage history from their smartphone. In some cases, this can be done so that the user can keep track of the timing and dosage for future use, and/or for the current administration, so that the user can know when to administer or deliver another micro-dose without being overly affected by taking too many doses. In some embodiments, a dispensing or delivery device can be configured for use with replaceable chambers, modules, and/or vials, and can be used more that once, and therefore be a multi-use device. A suite of different cannabinoid chambers, vials, and/or modules indicated or desired for various conditions can be provided to users or prescribed to patients. In some embodiments, a dispensing device may not include a touch pad, and may instead by be controlled by a dial that reports the units dosed to the smartphone application.

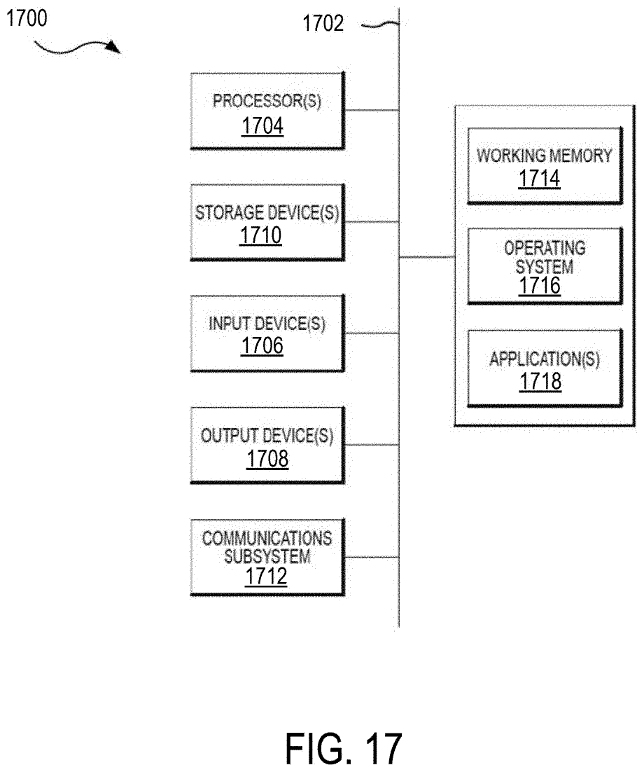

FIG. 17 depicts aspects of an exemplary computer system or device 1700 configured for use with any of the dispensing and/or mobile computing devices disclosed herein, according to embodiments of the present invention. An example of a computer system or device 1700 may include an enterprise server, blade server, desktop computer, laptop computer, tablet computer, personal data assistant, smartphone, any combination thereof, and/or any other type of machine configured for performing calculations. Any computing devices encompassed by embodiments of the present invention may be wholly or at least partially configured to exhibit features similar to the computer system 1700.

The computer system 1700 of FIG. 17 is shown comprising hardware elements that may be electrically coupled via a bus 1702 (or may otherwise be in communication, as appropriate). The hardware elements may include a processing unit with one or more processors 1704, including without limitation one or more general-purpose processors and/or one or more special-purpose processors (such as digital signal processing chips, graphics acceleration processors, and/or the like); one or more input devices 1706, which may include without limitation a remote control, a mouse, a keyboard, a keypad, a touchscreen, and/or the like; and one or more output devices 1708, which may include without limitation a presentation device (e.g., controller screen, display screen), a printer, and/or the like.

The computer system 1700 may further include (and/or be in communication with) one or more non-transitory storage devices 1710, which may comprise, without limitation, local and/or network accessible storage, and/or may include, without limitation, a disk drive, a drive array, an optical storage device, a solid-state storage device, such as a random access memory, and/or a read-only memory, which may be programmable, flash-updateable, and/or the like. Such storage devices may be configured to implement any appropriate data stores, including without limitation, various file systems, database structures, and/or the like.

The computer system 1700 can also include a communications subsystem 1712, which may include without limitation a modem, a network card (wireless and/or wired), an infrared communication device, a wireless communication device and/or a chipset such as a Bluetooth device, 802.11 device, WiFi device, WiMax device, cellular communication facilities such as GSM (Global System for Mobile Communications), W-CDMA (Wideband Code Division Multiple Access), LTE (Long Term Evolution), and the like. The communications subsystem 1712 may permit data to be exchanged with a network (such as the network described below, to name one example), other computer systems, controllers, and/or any other devices described herein. In many embodiments, the computer system 1700 can further comprise a working memory 1714, which may include a random access memory and/or a read-only memory device, as described above.

The computer system 1700 also can comprise software elements, shown as being currently located within the working memory 1714, including an operating system 1716, device drivers, executable libraries, and/or other code, such as one or more application programs 1718, which may comprise computer programs provided by various embodiments, and/or may be designed to implement methods, and/or configure systems, provided by other embodiments, as described herein. By way of example, one or more procedures described with respect to the method(s) discussed herein, and/or system components might be implemented as code and/or instructions executable by a computer (and/or a processor within a computer); in an aspect, then, such code and/or instructions may be used to configure and/or adapt a general purpose computer (or other device) to perform one or more operations in accordance with the described methods.

A set of these instructions and/or code can be stored on a non-transitory computer-readable storage medium, such as the storage device(s) 1710 described above. In some cases, the storage medium might be incorporated within a computer system, such as computer system 1700. In other embodiments, the storage medium might be separate from a computer system (e.g., a removable medium, such as flash memory), and/or provided in an installation package, such that the storage medium may be used to program, configure, and/or adapt a general purpose computer with the instructions/code stored thereon. These instructions might take the form of executable code, which is executable by the computer system 1700 and/or might take the form of source and/or installable code, which, upon compilation and/or installation on the computer system 1700 (e.g., using any of a variety of generally available compilers, installation programs, compression/decompression utilities, and the like), then takes the form of executable code.

It is apparent that substantial variations may be made in accordance with specific requirements. For example, customized hardware might also be used, and/or particular elements might be implemented in hardware, software (including portable software, such as applets, and the like), or both. Further, connection to other computing devices such as network input/output devices may be employed.

As mentioned elsewhere herein, in one aspect, some embodiments may employ a computer system (such as the computer system 1700) to perform methods in accordance with various embodiments of the disclosure. According to a set of embodiments, some or all of the procedures of such methods are performed by the computer system 1700 in response to processor 1704 executing one or more sequences of one or more instructions (which might be incorporated into the operating system 1716 and/or other code, such as an application program 1718) contained in the working memory 1714. Such instructions may be read into the working memory 1714 from another computer-readable medium, such as one or more of the storage device(s) 1710. Merely by way of example, execution of the sequences of instructions contained in the working memory 1714 may cause the processor(s) 1704 to perform one or more procedures of the methods described herein.

The terms “machine-readable medium” and “computer-readable medium,” as used herein, can refer to any non-transitory medium that participates in providing data that causes a machine to operate in a specific fashion. In an embodiment implemented using the computer system 1700, various computer-readable media might be involved in providing instructions/code to processor(s) 1704 for execution and/or might be used to store and/or carry such instructions/code. In many implementations, a computer-readable medium is a physical and/or tangible storage medium. Such a medium may take the form of a non-volatile media or volatile media. Non-volatile media may include, for example, optical and/or magnetic disks, such as the storage device(s) 1710. Volatile media may include, without limitation, dynamic memory, such as the working memory 1714.

Exemplary forms of physical and/or tangible computer-readable media may include a floppy disk, a flexible disk, hard disk, magnetic tape, or any other magnetic medium, a compact disc, any other optical medium, ROM, RAM, and the like, any other memory chip or cartridge, or any other medium from which a computer may read instructions and/or code. Various forms of computer-readable media may be involved in carrying one or more sequences of one or more instructions to the processor(s) 1704 for execution. By way of example, the instructions may initially be carried on a magnetic disk and/or optical disc of a remote computer. A remote computer might load the instructions into its dynamic memory and send the instructions as signals over a transmission medium to be received and/or executed by the computer system 1700.

The communications subsystem 1712 (and/or components thereof) generally can receive signals, and the bus 1702 then can carry the signals (and/or the data, instructions, and the like, carried by the signals) to the working memory 1714, from which the processor(s) 1704 retrieves and executes the instructions. The instructions received by the working memory 1714 may optionally be stored on a non-transitory storage device 1710 either before or after execution by the processor(s) 1704.

It should further be understood that the components of computer system 1700 can be distributed across a network. For example, some processing may be performed in one location using a first processor while other processing may be performed by another processor remote from the first processor. Other components of computer system 1700 may be similarly distributed. As such, computer system 1700 may be interpreted as a distributed computing system that performs processing in multiple locations. In some instances, computer system 1700 may be interpreted as a single computing device, such as a distinct laptop, desktop computer, or the like, depending on the context.

A processor may be a hardware processor such as a central processing unit (CPU), a graphic processing unit (GPU), or a general-purpose processing unit. A processor can be any suitable integrated circuits, such as computing platforms or microprocessors, logic devices and the like. Although the disclosure is described with reference to a processor, other types of integrated circuits and logic devices are also applicable. The processors or machines may not be limited by the data operation capabilities. The processors or machines may perform 512 bit, 256 bit, 128 bit, 64 bit, 32 bit, or 16 bit data operations.

Each of the calculations or operations discussed herein may be performed using a computer or other processor having hardware, software, and/or firmware. The various method steps may be performed by modules, and the modules may comprise any of a wide variety of digital and/or analog data processing hardware and/or software arranged to perform the method steps described herein. The modules optionally comprising data processing hardware adapted to perform one or more of these steps by having appropriate machine programming code associated therewith, the modules for two or more steps (or portions of two or more steps) being integrated into a single processor board or separated into different processor boards in any of a wide variety of integrated and/or distributed processing architectures. These methods and systems will often employ a tangible media embodying machine-readable code with instructions for performing the method steps described herein. All features of the described systems are applicable to the described methods mutatis mutandis, and vice versa. Suitable tangible media may comprise a memory (including a volatile memory and/or a non-volatile memory), a storage media (such as a magnetic recording on a floppy disk, a hard disk, a tape, or the like; on an optical memory such as a CD, a CD-R/W, a CD-ROM, a DVD, or the like; or any other digital or analog storage media), or the like. While the exemplary embodiments have been described in some detail, by way of example and for clarity of understanding, those of skill in the art will recognize that a variety of modification, adaptations, and changes may be employed.

According to some embodiments, machine-readable code instructions for, and/or data generated or used by, dispensing devices and/or computing devices (which may include smart phones or other mobile computing devices) can be stored on or executed by any of a variety of computing modalities, including without limitation personal computers, servers (e.g. hosted and/or privately owned servers), internet connections, cloud hosts, cloud based storage, and the like. According to some embodiments, data acquired by a dispensing device can be related to a mobile computing device (e.g. a smart phone) via a data exchange technology (e.g. Bluetooth) and then relayed to a secure server via the cloud (e.g. servers that are accessed over the Internet, and the software, services, and/or databases that run on those servers). In some embodiments, the data can be acquired in alignment with the Health Insurance Portability and Accountability Act of 1996 (HIPAA) regulations. Once the data is acquired it can be stored on a secure and HIPPA compliant server. The acquisition of this data can be in a metric format that can be married with observational, ailment specific, and strain specific data via an application programming interface (API) interface. In some embodiments, the dispensing devices enable the creation of authentic user data that can be integrated into and used within and without an artificial intelligence system of data aggregation to further the understanding and benefits of Cannabis and its formulations for medicinal benefits. In some instances, a mobile computing application can be configured for use by individuals in the Medical/Wellness Cannabis space. The application can work in conjunction with an oil or preparation dispensing pen or device that the patients use to medicate. In order for patients to know how much of a particular oil or preparation to dose, the application can include a dosing calculator icon that determines how a setting on the dispensing device can correspond to a particular milligram dose of the active ingredients in the oil or preparation and records the date, time and setting configuration of that calculation. A feedback reports icon can provide the user with a graph that shows the user a calculation history according to selected time frame (e.g. week, month, 3 months, 6 months, year) to display patterns of use history. Other features of an application can include a links icon where a user can find links to a dosing guidelines published by peer reviewed and medical doctors (informational only), industry Partners, and a Find us “where to buy” page. In some embodiments, an application can be configured for use within another Cannabis symptoms to dose tracking application. The application and dosing calculator can be configured for use with other user interface (UI) Cannabis applications, and the application can be used with a dispensing device as a trackable dosing application. In some embodiments, anytime the calculator is used via this application or another application, the data is still recorded and sent directly back to a database. In exemplary embodiments, a user may be a patient looking for a precise, controlled, and consistent method for oral consumption of Cannabis whole plant oils or tinctures. A user may be sensitive to THC or are fearful of oral dosing methods (e.g. edibles) and of taking too much and realize small doses may help. The user can open the application and enter their profile information. Once that is completed the user is taken to the home screen where they will see the dose calculator, Reports, and Links. A dosing guidelines link, sites of partners and where to purchase the dispensing devices and/or vials, may also be included. The application can allows the user to provide the product information and enter the THC to CBD (cannabinoid) ratios and their recommended dose. The calculator can automatically convert those ratios into settings for the dispensing device, giving the user a precise way to dose their medicine. User can track the history of their doses and use that information to make adjustments if needed. An administrator can log into a server to download the secured data captured from each user. That data can be used to print reports, graphs or to make future recommendations on how the application should be used. A data scientist can organize this data in such a way that it is valuable to researchers and product formulators in the industry. The data can flow into a database and an algorithm can enable the generation of automated feedback reports based on the controls outlined. According to some embodiments, an individual can use the application by entering a username and password, entering user profile data (e.g. via Set up) that flows into control fields used for data analysis. The user can also enter gender, age (e.g. date or birth), and/or email information. A user can select a dosing calculator (e.g. via a home screen), and enter in product information used in the dose calculator. The user can use the application to calculate a desired dose in milligrams and number of dose sessions per day, and to calculate the settings for the dispensing device to achieve desired dose. Each time a user uses the dose calculator the application can record the date, time, and settings or dispensed amount. A user can select a reports icon to generate a feedback report chart showing the number of doses and/or dosing amount settings over a period of time selected by the user (e.g. week, month, 3 months, 6 months, year). A mobile application can enable a user to download dosing history to a spreadsheet such as an Excel document, using a report icon, and the report can be automatically sent to the user email address provided in the profile. A user can use a links icon to access links to dosing guidelines published by peer reviewed and medical doctors (informational only), industry partners, and/or a find us “where to buy” page. In some embodiments, an administrator, which may be an application administrator, can logs into a secured server, and have access to back office and all data controls through an administrator user interface portal accessible via a virtual private network (VPN) web login. An administrator can download all data that has been inputted by users, e.g. via a set controlled report features developed in the administrator user interface. This can also allow an administrator search or filter data based on preset data filter settings. In some embodiments, data filter settings can include (e.g. filter by) gender, age, number of dose sessions per day, product name, product type, and/or milligrams dosed (or related device setting). In some embodiments, an administrator can use the data to recommend or implement future application development improvements. In some embodiments, an application can have a dose calculator running in the background that can determine milligrams per milliliter of a particular cannabinoid (e.g. based on information provided by a user and/or manufacturer of oils or preparations). The application can take this information and convert it to a settings designation for the dispensing device, so as to achieve a certain amount of milligrams per dose. The application can operate to collect the user data and to provide historical dosing reports as described elsewhere herein. The application can also provide graphs or charts displaying the users history and progress of each dose taken. According to some application embodiments, a user can be greeted with a registration screen for the first time the application is downloaded. The registration screen can enable the user to establish an account username and password, to accept terms and conditions (which may include a release of liability of HIPAA compliance), to set up user profile providing data that flows into control fields used for data analysis, to indicate gender, age (e.g. date of birth), and email information. In some embodiments, a home screen of an application can provide icons for a dose calculator and/or a health journal (e.g. notification and response input feature). A dosing calculator can enable to a user to select or input certain dosing information, as disclosed elsewhere herein. In some cases, a user can select a product from a scroll down menu. In some cases, if the user selected the other option, then the other form screen would be displayed. This could be a basic form that allows for the user to enter certain information. Such information can be approved on the back end for its accuracy before added to the options in the product name. According to some embodiments, for an initial dose session that the “other” form was initiated, it may be desirable to be able to use the numeric entries for the ml values and provide the user a dose conversion (e.g. ml/mg to device number settings). Once it is approved by the administrator it can flow into the database available for selection by other users in the future. According to some embodiments, based on the product and Cannabis values selected, the user can then be prompted to select the “activate dose calculator” as disclosed elsewhere herein. In some embodiments, mobile application displays can allow the user to select the desired milligram amount which will then provide the device setting (e.g. number) to achieve the desired dose. In some embodiments, the calculator can operate to calculate either a THC dose or CBD dose (not BOTH), and for the cannabinoid not calculated an adjustment can be made based on the THC:CBD ratio.

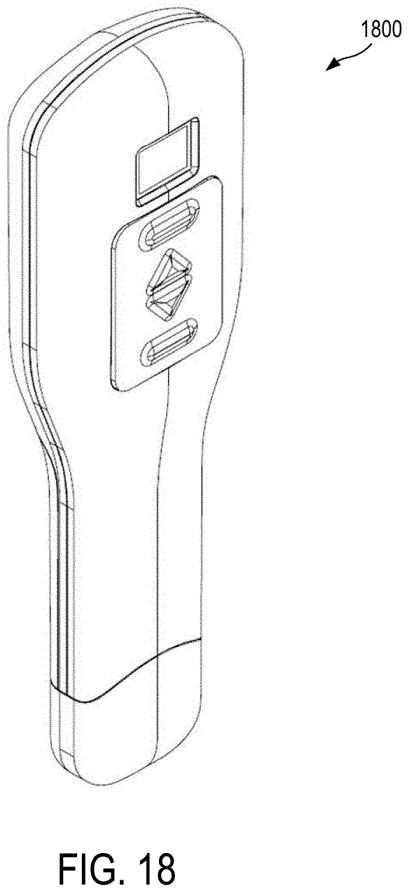

FIG. 18 depicts aspects of a chambered dispensing device 1800 according to embodiments of the present invention. In some cases, dispensing device 1800 may be provided as a multi-chamber dispensing device. In some cases, dispensing device 1800 can include two compound or preparation delivery vials.

FIG. 19 depicts aspects of a chambered dispensing device 1900 according to embodiments of the present invention. As shown here, the dispensing device 1900 can be a multi-chamber dispensing device, having multiple compound or preparation delivery vials or chambers 1901. For example, dispensing device 1900 may include two preparation vials 1901. In some embodiments, vials 1901 can have one or more features of vials such as those disclosed in previously incorporated U.S. patent application Ser. No. 16/712,264 filed Dec. 12, 2019. The vials 1901 can be connected to or otherwise in operational engagement with stepper motors or other drive mechanisms 1902 (e.g. one vial in connectivity with one respective stepper motor). The dispensing device 1900 can also include a plunger 1903 at the end of, or otherwise in operational engagement with, a respective stepper motor 1902. In use, a stepper motor can operate to actuate a plunger, so as to move a stopper 1913 and dispense an amount of a preparation from a vial. In some embodiments, stoppers 1913 can have one or more features of stoppers such as those disclosed in previously incorporated U.S. patent application Ser. No. 16/712,264 filed Dec. 12, 2019. Dispensing device 1900 also includes a circuit board assembly or computing system 1904, which may include a microprocessor controlled circuit, and may be in operational connectivity with one or more stepper motors, and other components of the dispensing device 1900 described herein. Dispensing device 1900 also includes a front housing 1906, a rear housing 1907, a rear plate 1905, and dispensing tips 1908. In use, the rear plate 1905 can be reversibly removed from rear housing 1907, so as to facilitate the easy and efficient replacement of vials and/or dispenser tips. In some embodiments, dispensing tips 1908 can have one or more features of dispensing tips such as those disclosed in previously incorporated U.S. patent application Ser. No. 16/712,264 filed Dec. 12, 2019. Dispensing device 1900 also includes an end cap 1909, an indicator screen 1910, adjustment UP and DOWN keypad buttons 1911, a dispensing button 1912, and an ON/OFF button 1913. In some embodiments, the dispenser tips 1908 can be configured to receive one or more amounts of expelled preparation from one or more of the vials (e.g. via one or more inlet conduits), and to dispense the one or more amounts of preparation (e.g. via an outlet conduit).

The front housing 1906 and rear housing 1907 can cover the circuit board assembly or computing system 1904 and the stepper motors 1902. The end cap 1909 can cover the dispenser tips 1908 after each use (e.g. dispensing of preparation). In some cases, the front and rear housing can form at least part of a connecting body, and the connecting body may include a location or holding container for one, two, three, or more separate reservoirs or chambers.

Externally actuation of the ON/OFF button 1913 can operate to control the power of the dispensing device 1900, which in some embodiments may time out after 30 seconds of non-use. The indicator screen 1910 can be configured to show the amount of the dose as chosen by the user via actuation of the UP and DOWN keypad buttons 1911. The dispensing button 1912 can operate to activate the dispensing process which can precisely control one or more chambers of the preparation(s).

A user can select the preparation dosage via one or more of the UP/DOWN keypad buttons 1911, and setting the dose of a preparation by a user can give an individual or patient more reliable control over a desired or therapeutic outcome of micro-dosed treatment or therapy. In some embodiments, a combined variable dose treatment or therapy may be preferable over a single dose treatment or therapy. In some embodiments, a preparation or compound dispensing interface 1908 can include a hollow spout for the delivery dispensing of fluid or liquid preparations or compounds. In some embodiments, the dispensing device 1900 may include a touch pad interface for simple UP and DOWN control of the dispensing or dosing device.

The circuit board assembly or computing system 1904 can be in operational connectivity with one or more of the stepper motor(s) 1902, indicator screen 1910, adjustment UP and DOWN keypad buttons 1911, dispensing button 1912, and/or ON/OFF button 1913. In use, the computing system 1904 can receive input signals or data from adjustment UP and DOWN keypad buttons 1911, dispensing button 1912, and/or ON/OFF button 1913, and can provide output signals or data to one or more of the stepper motor(s) 1902 and/or indicator screen 1910.

A multi-chamber medicament dispensing device 1900 can have medicament modules/vials 1901, and dispenser tips 1908. The dispensing device 1900 can be controlled electronically. In some cases, the modules or vials 1901 may be interchanged and replaced with various medicaments. In some cases, the modules or vials 1901 may provide a precise volume of medicaments. In some cases, the modules or vials 1901 may provide a precise volume of cannabis-derived medicaments. In some cases, the dispensing device 1900 is configured to be electronically tracked, mechanical, and non-motorized.

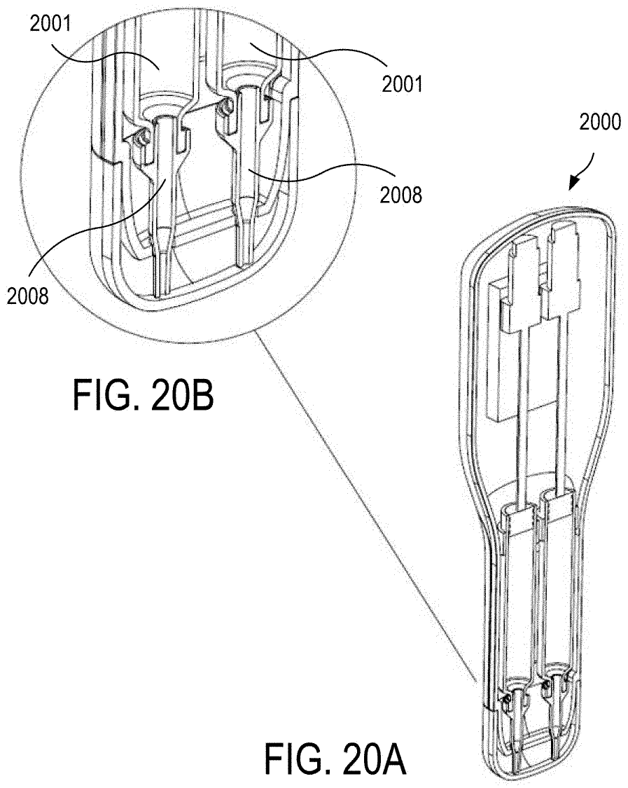

FIG. 20A provides a cross-section view of a portion of a dispensing device 2000. FIG. 20B depicts a close-up view of the engagement or interface between the vials 2001 and the dispenser tips 2008. In some embodiments, the engagement or interface between the vials 2001 and dispenser tips 2008 can have one or more features of the engagement or interface between vials and dispenser tips such as those disclosed in previously incorporated U.S. patent application Ser. No. 16/712,264 filed Dec. 12, 2019.

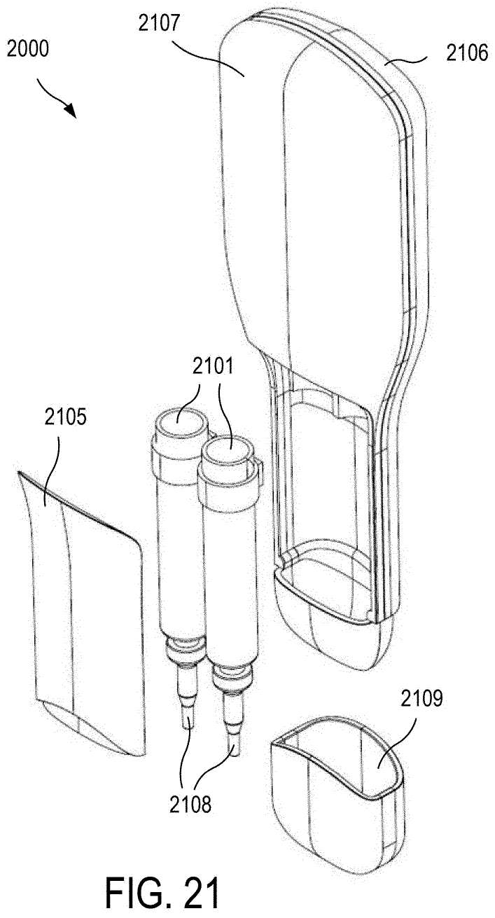

FIG. 21 provides a cross-section view of a portion of a dispensing device 2100. Dispensing device 2100 includes a front housing 2106, a rear housing 2107, a rear plate 2105, vials 2101, dispensing tips 2108, and an end cap 2109. In use, the rear plate 2105 can be reversibly removed from rear housing 2107, so as to facilitate the easy and efficient replacement of vials 2101 and/or dispenser tips 2108.

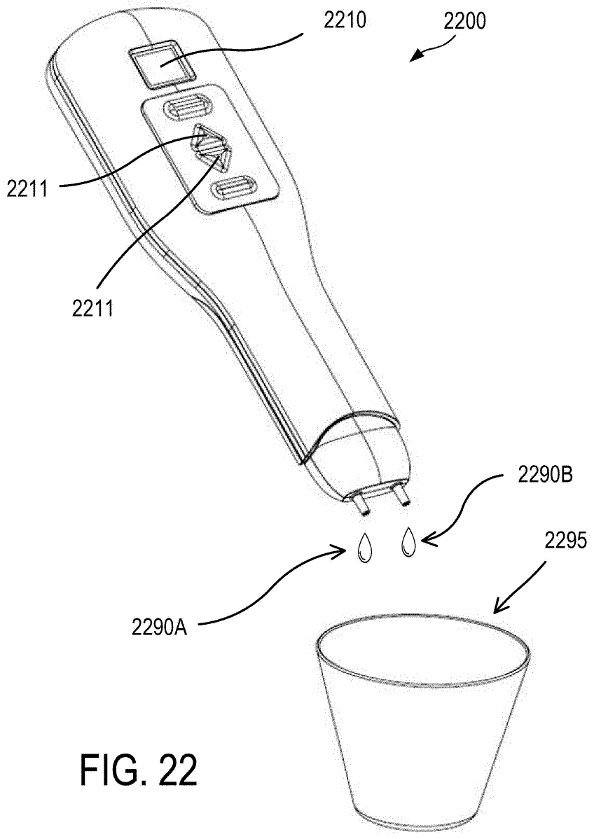

FIG. 22 depicts aspects of a chambered dispensing device 2200 according to embodiments of the present invention. As shown here, the dispensing device 2200 can be a multi-chamber dispensing device, having multiple (e.g. two) compound or preparation delivery vials or chambers. The device 2200 can be used with the vials (e.g. fluid or liquid compound vials) to spray or dispense fluid or liquid into a beverage container. The embodiment depicted in FIG. 22 provides an exemplary layout of a housing face view of a dispensing device showing the UP and DOWN dose control buttons with an indicator display. As shown here, dispensing device 2200 can be used to dispense one or more amounts of one or more preparations 2290A, 2290B therefrom, for example into a container or receptacle 2295 such as a drinking glass. In some embodiments, an amount of preparation 2290A can include a tincture or preparation containing CBD with very little or no THC. Exemplary dominant CBD tinctures or preparations include 20:1 to 30:1 CBD to THC ratios. In some embodiments, an amount of preparation 2290B can include a tincture containing THC with very little or no CBD. Exemplary dominant THC tinctures or preparations include 10:1 to 2:1 THC to CBD ratios. A user can operate the device 2200 (e.g. using the UP/DOWN arrow keys 2211) so as to designate a desired number of milligrams of THC and/or CBD (e.g. from respective vials or chambers containing the preparations) for dispensing from the device 2200. For example, a user can operate the device 2200 to dispense an amount of preparation (which may be a combined preparation) having a 1:1 ratio of THC and CBD. Dispensing device embodiments disclosed herein can be configured to dispense other types of materials that are not related to Cannabis, such as beauty and cosmetic preparations, medical preparations, culinary preparations, homeopathic preparations, whole plant preparations, plant extract preparations, medicament preparations, and other preparations discussed elsewhere herein. Dispensing device embodiments disclosed herein can be configured to dispense Cannabis related materials that are not THC or CBD, such as CBN and other cannabinoid preparations discussed elsewhere herein. Dispensing device embodiments disclosed herein can be configured to dispense any other psychoactive/psychotropic and/or non-psychoactive/non-psychotropic materials.

As shown here, dispensing device 2200 can include UP and DOWN dose control buttons 2211 and a display 2210, such as a numbered LED display or indicator screen. Further, dispensing device 2200 can include one or more dispensing or GO buttons, and an ON/OFF button. In operations, a dose for dispensing from the dispensing device 2200 can be set using one or more of the dose setter buttons 2211, e.g. by selecting an appropriate number of units or incremental amounts of preparation. Dispensation of the fluid or liquid compound or preparation may then be achieved by dispensing the preparation (e.g. water soluble liquid contents) via activation of the GO button on device 2200. In some embodiments, the dose buttons 2211 are operably connected to a printed circuit board (PCB) assembly or computing system (e.g. computing system 204 depicted in FIG. 2) that engages one or more stepper motors to push on reservoirs of each vial containing the fluid or liquid preparations.

Dispensing device 2200 can be used with liquid compound vials for spraying liquid contents into a beverage container. As shown here, dispenser device 2200 can include UP and DOWN dose control buttons and an indicator display. According to some embodiments, a dispenser device 2200 can include dose setter buttons, and a dose to be dispensed by the dispensing device can be set or selected by using the dose setting buttons, for example by selecting an appropriate number of units. Dispensing of the liquid compounds can be achieved by dispensing water soluble liquid contents from the dispensing device via activation of a GO button on the device. In some embodiments, the dose buttons can be operably connected with a printed circuit board (PCB) assembly that engages or is in otherwise operational connectivity with each stepper motor, to push on reservoirs of one or more vial containing liquid compounds.

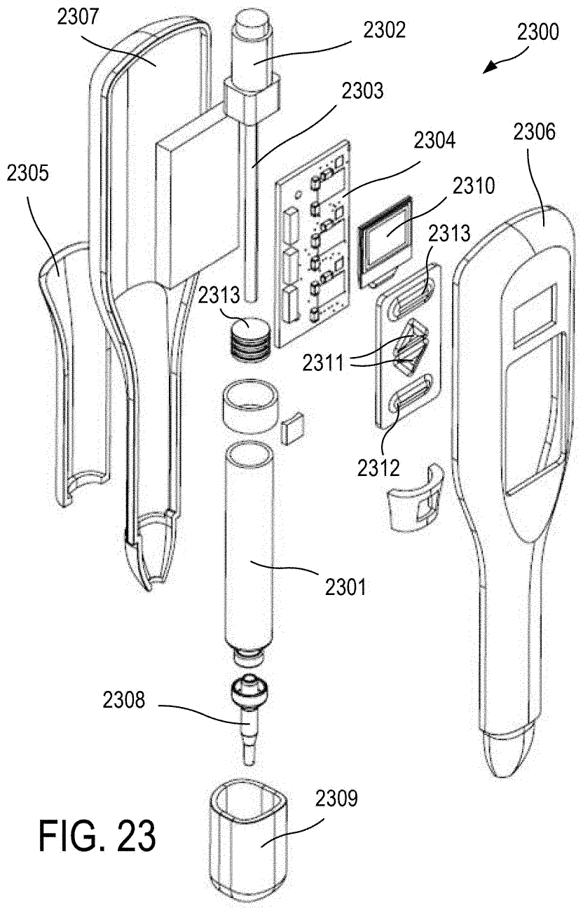

FIG. 23 depicts aspects of a chambered dispensing device 2300 according to embodiments of the present invention. As shown here, the dispensing device 2300 can be a single chamber dispensing device, having a compound or preparation delivery vial or chamber 2301. In some embodiments, vial 2301 can have one or more features of a vial such as that which is disclosed in previously incorporated U.S. patent application Ser. No. 16/712,264 filed Dec. 12, 2019. The vial 2301 can be connected to or otherwise in operational engagement with a stepper motor or other drive mechanism 2302 (e.g. one vial in connectivity with one respective stepper motor). The dispensing device 2300 can also include a plunger 2303 at the end of, or otherwise in operational engagement with, a respective stepper motor 2302. In use, a stepper motor can operate to actuate a plunger, so as to move a stopper 2313 and dispense an amount of a preparation from a vial. In some embodiments, a stoppers 2313 can have one or more features of a stopper such as that which is disclosed in previously incorporated U.S. patent application Ser. No. 16/712,264 filed Dec. 12, 2019. Dispensing device 2300 also includes a circuit board assembly or computing system 2304, which may include a microprocessor controlled circuit, and may be in operational connectivity with one or more stepper motors, and other components of the dispensing device 2300 described herein. Dispensing device 2300 also includes a front housing 2306, a rear housing 2307, a rear plate 2305, and a dispensing tip 2308. In use, the rear plate 2305 can be reversibly removed from rear housing 2307, so as to facilitate the easy and efficient replacement of the vials and/or dispenser tip. In some embodiments, a dispensing tip 2308 can have one or more features of a dispensing tip such as that which is disclosed in previously incorporated U.S. patent application Ser. No. 16/712,264 filed Dec. 12, 2019. Dispensing device 2300 also includes an end cap 2309, an indicator screen 2310, adjustment UP and DOWN keypad buttons 2311, a dispensing button 2312, and an ON/OFF button 2313. In some embodiments, the dispenser tip 2308 can be configured to receive one or more amounts of expelled preparation from a vial (e.g. via an inlet conduit), and to dispense the amount of preparation (e.g. via an outlet conduit).

The front housing 2306 and rear housing 2307 can cover the circuit board assembly or computing system 2304 and the stepper motor 2302. The end cap 2309 can cover the dispenser tip 2308 after each use (e.g. dispensing of preparation). In some cases, the front and rear housing can form at least part of a connecting body, and the connecting body may include a location or holding container for one, two, three, or more separate reservoirs or chambers.

Externally actuation of the ON/OFF button 2313 can operate to control the power of the dispensing device 2300, which in some embodiments may time out after 30 seconds of non-use. The indicator screen 2310 can be configured to show the amount of the dose as chosen by the user via actuation of the UP and DOWN keypad buttons 2311. The dispensing button 2312 can operate to activate the dispensing process which can precisely control one or more chambers of the preparation(s).

A user can select the preparation dosage via one or more of the UP/DOWN keypad buttons 2311, and setting the dose of a preparation by a user can give an individual or patient more reliable control over a desired or therapeutic outcome of micro-dosed treatment or therapy. In some embodiments, a combined variable dose treatment or therapy may be preferable over a single dose treatment or therapy. In some embodiments, a preparation or compound dispensing interface 2308 can include a hollow spout for the delivery dispensing of fluid or liquid preparations or compounds. In some embodiments, the dispensing device 2300 may include a touch pad interface for simple UP and DOWN control of the dispensing or dosing device.

The circuit board assembly or computing system 2304 can be in operational connectivity with the stepper motor 2302, indicator screen 2310, adjustment UP and DOWN keypad buttons 2311, dispensing button 2312, and/or ON/OFF button 2313. In use, the computing system 2304 can receive input signals or data from adjustment UP and DOWN keypad buttons 2311, dispensing button 2312, and/or ON/OFF button 2313, and can provide output signals or data to the stepper motor 2302 and/or indicator screen 2310.

A single chamber medicament dispensing device 2300 can have a medicament module/vial 1901, and a dispenser tip 2308. The dispensing device 2300 can be controlled electronically. In some cases, the module or vial 2301 may be interchanged and replaced with various medicaments. In some cases, the module or vial 2301 may provide a precise volume of medicaments. In some cases, the module or vial 2301 may provide a precise volume of cannabis-derived medicaments. In some cases, the dispensing device 2300 is configured to be electronically tracked, mechanical, and non-motorized.

FIG. 24A provides a cross-section view of a portion of a dispensing device 2400. FIG. 24B depicts a close-up view of the engagement or interface between the vial 2401 and the dispenser tip 2408. In some embodiments, the engagement or interface between the vial 2301 and dispenser tip 2308 can have one or more features of the engagement or interface between a vial and dispenser tip such as those disclosed in previously incorporated U.S. patent application Ser. No. 16/712,264 filed Dec. 12, 2019.

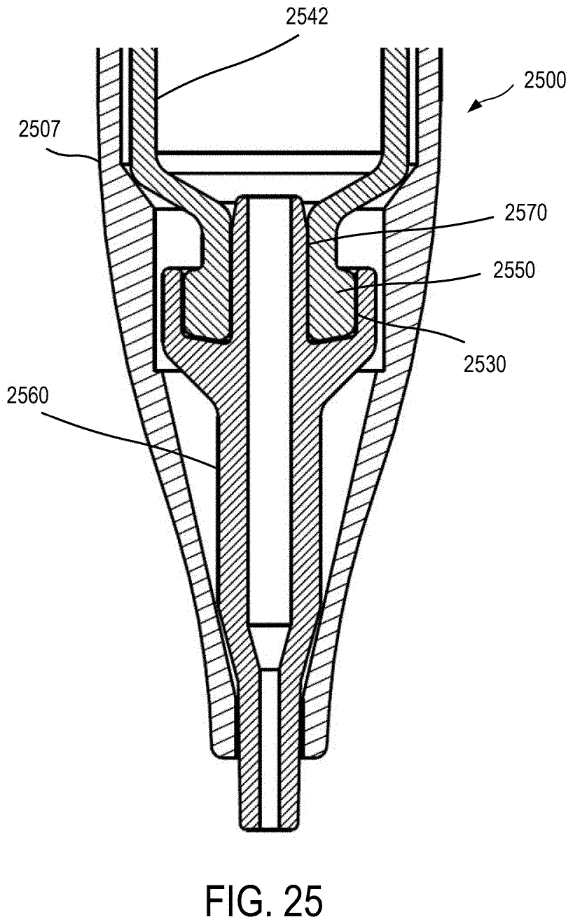

FIG. 25 provides a cross-section view of a portion of a dispensing device 2500, according to embodiments of the present invention. As shown here, when dispenser tip 2560 and vial 2542 are engaged with one another, a leak-tight junction 2570 therebetween is formed. In some embodiments, dispenser tip 2560 may include an inside groove 2530. The groove 2530 can be configured to mate to and against a molded rim 2550 of the vial 2542. In some cases, the inside groove 2530 is part of a snap-on cap of the dispenser tip 2560. In some cases, the snap-on cap can be configured to slip-over and lock onto the rim 2550. In some cases, the dispenser tip 2560 and the vial 2542 are joined together by a snap-on mating of rim 2550 of vial 2542, and the snap-on cap of dispenser tip 2560, the snap-on connection holds pressure on the associated or apposed surfaces of the dispenser tip 2560 and vial 2542 at junction or interface 2570, and the pressure in turn maintains the leak-tight seal of a cartridge subassembly. As shown here, the dispenser tip 256 and vial 2542 can be positioned or contained within a body of the dispensing device, such as within a rear housing 2507 and/or front housing.

According to some embodiments, there may be a slight size differential between the outer diameter of the vial rim 2550 and the inner surface of the dispenser tip groove 2530, such that a press fit, friction fit, or interference fit between the vial 2542 and the dispenser tip 2560 is created when the vial 2542 and dispenser tip 2560 are engaged with one another. According to some embodiments, there may be a slight size differential between the surface of the vial at interface 2570 and the surface of the dispenser tip at interface 2570, such that a press fit, friction fit, or interference fit between the vial 2542 and the dispenser tip 2560 is created when the vial 2542 and dispenser tip 2560 are engaged with one another. As a result, the press fit, friction fit, or interference can operate to resist separation of the engaged vial 2542 and dispenser tip 2560. In some cases, when the vial 2542 and dispenser tip 2560 are pressed together, a press fit, a friction fit, or an interference fit is formed therebetween.

It was surprisingly discovered that when the distal nozzle of the dispenser tip extends distally from the viewing assembly, as shown in FIG. 25, there was a reduction in the amount of migration of the dispensed preparation material as it exited the exit port of the distal nozzle, as compared to configurations where the distal end of the distal nozzle was flush with the distal end of the viewing assembly. When the distal end of the distal nozzle was flush with the distal end of the viewing assembly, it was observed that a portion of the dispensed preparation material could tend to migrate from the distal nozzle to the viewing assembly, and thereby interfere with the desired dispensing action of the dispenser device. It was observed, for example, that a distal nozzle that extended 1/16 or 1/32 of an inch past the distal end of the viewing assembly results in a reduction or elimination of the migrating preparation material. Embodiments of the present invention encompass dispenser tips having any of a variety of desired sizes and/or gauges. In some cases, the size and/or gauge may be selected based on the consistency or viscosity of preparation to be dispensed. For example, with a more viscous preparation material, which may be a Cannabis dab material, a Cannabis gel material, or a Cannabis honey material, a dispenser tip with a wider gauge distal nozzle may be used (e.g. as compared to a dispenser tip for use with a less viscous Cannabis oil preparation material). Relatedly, with more viscous preparation materials, it may be advantageous to use a dispenser tip having a metal (e.g. stainless steel) distal nozzle instead of a plastic distal nozzle. Dispenser tips having metal distal nozzles can also be used with preparation materials which may benefit from heating prior to dispensing, such as Cannabis dab preparations.

What is more, as depicted in FIG. 25, the proximal end of the dispenser tip is generally flush or aligned with the distal portion of the main body or chamber of the vial. In this way, the dispenser tip allows most or all of the preparation material to be dispensed, rather than having significant amounts of preparation material settle and/or remain in a location within the vial and circumferentially peripheral and distal to the proximal port of the dispenser tip.

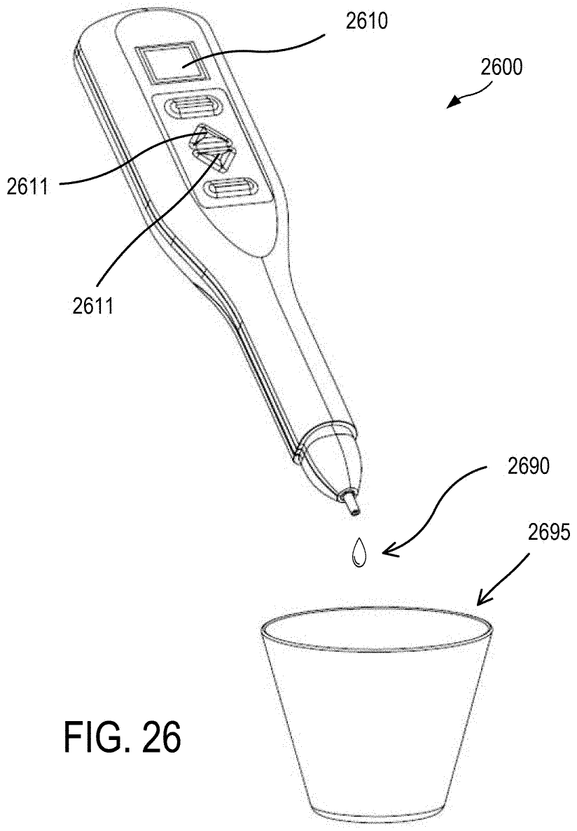

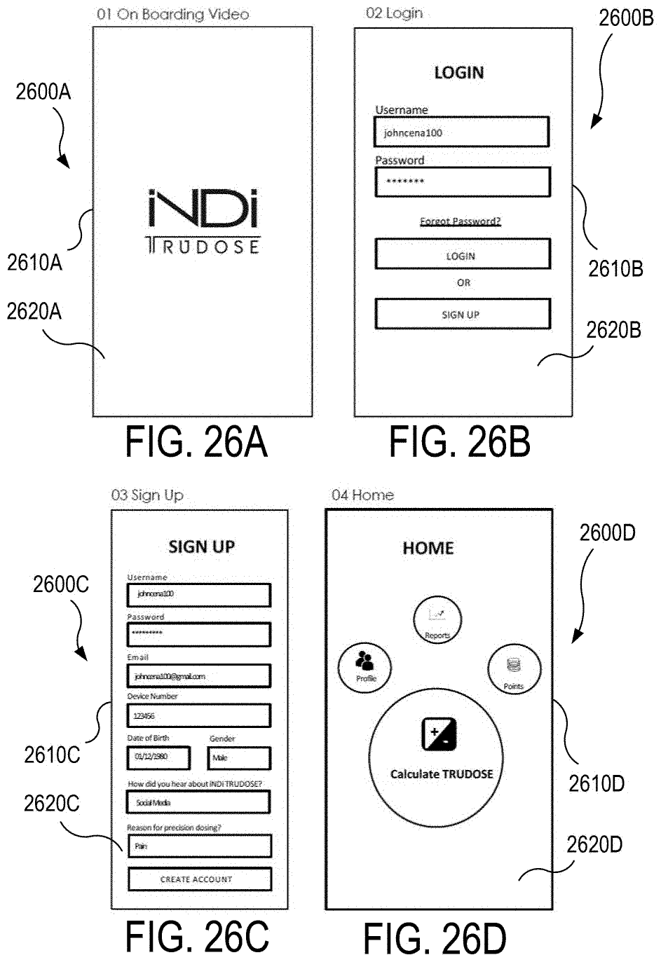

FIG. 26 depicts aspects of a chambered dispensing device 2600 according to embodiments of the present invention. As shown here, the dispensing device 2600 can be a single chamber dispensing device, having one compound or preparation delivery vial or chamber. The device 2600 can be used with the vial (e.g. fluid or liquid compound vial) to spray or dispense fluid or liquid into a beverage container. The embodiment depicted in FIG. 26 provides an exemplary layout of a housing face view of a dispensing device showing the UP and DOWN dose control buttons with an indicator display. As shown here, dispensing device 2600 can be used to dispense an amount of a preparation 2690 therefrom, for example into a container or receptacle 2695 such as a drinking glass. In some embodiments, an amount of preparation 2690 can include a tincture or preparation containing CBD with very little or no THC. Exemplary dominant CBD tinctures or preparations include 20:1 to 30:1 CBD to THC ratios. In some embodiments, an amount of preparation 2690 can include a tincture containing THC with very little or no CBD. Exemplary dominant THC tinctures or preparations include 10:1 to 2:1 THC to CBD ratios. A user can operate the device 2600 (e.g. using the UP/DOWN arrow keys 2211) so as to designate a desired number of milligrams of THC and/or CBD (e.g. from respective vials or chambers containing the preparations) for dispensing from the device 2600. For example, a user can operate the device 2600 to dispense an amount of preparation (which may be a combined preparation) having a 1:1 ratio of THC and CBD. Dispensing device embodiments disclosed herein can be configured to dispense other types of materials that are not related to Cannabis, such as beauty and cosmetic preparations, medical preparations, culinary preparations, homeopathic preparations, whole plant preparations, plant extract preparations, medicament preparations, and other preparations discussed elsewhere herein. Dispensing device embodiments disclosed herein can be configured to dispense Cannabis related materials that are not THC or CBD, such as CBN and other cannabinoid preparations discussed elsewhere herein. Dispensing device embodiments disclosed herein can be configured to dispense any other psychoactive/psychotropic and/or non-psychoactive/non-psychotropic materials.

As shown here, dispensing device 2600 can include UP and DOWN dose control buttons 2611 and a display 2610, such as a numbered LED display or indicator screen. Further, dispensing device 2600 can include one or more dispensing or GO buttons, and an ON/OFF button. In operations, a dose for dispensing from the dispensing device 2600 can be set using one or more of the dose setter buttons 2611, e.g. by selecting an appropriate number of units or incremental amounts of preparation. Dispensation of the fluid or liquid compound or preparation may then be achieved by dispensing the preparation (e.g. water soluble liquid contents) via activation of the GO button on device 2600. In some embodiments, the dose buttons 2611 are operably connected to a printed circuit board (PCB) assembly or computing system (e.g. computing system 204 depicted in FIG. 2) that engages one or more stepper motors to push on reservoirs of each vial containing the fluid or liquid preparations.

Dispensing device 2600 can be used with a liquid compound vial for spraying liquid contents into a beverage container. As shown here, dispenser device 2600 can include UP and DOWN dose control buttons and an indicator display. According to some embodiments, a dispenser device 2600 can include dose setter buttons, and a dose to be dispensed by the dispensing device can be set or selected by using the dose setting buttons, for example by selecting an appropriate number of units. Dispensing of the liquid compounds can be achieved by dispensing water soluble liquid contents from the dispensing device via activation of a GO button on the device. In some embodiments, the dose buttons can be operably connected with a printed circuit board (PCB) assembly that engages or is in otherwise operational connectivity with each stepper motor, to push on reservoirs of one or more vial containing liquid compounds.

FIGS. 26A to 32B depict aspects of user flow embodiments that can be implemented via a graphical user interface, for example of a mobile computing device such as a smart phone.

FIG. 26A depicts aspects of a mobile computing device 2600A having a display screen 2610A with a graphical user interface 2620A. As shown here, user interface 2620A includes a presentation of an on boarding video.

FIG. 26B depicts aspects of a mobile computing device 2600B having a display screen 2610B with a graphical user interface 2620B. As shown here, user interface 2620B includes a presentation of a login or sign up screen.

FIG. 26C depicts aspects of a mobile computing device 2600C having a display screen 2610C with a graphical user interface 2620C. As shown here, user interface 2620C includes a presentation of a sign up screen.

FIG. 26D depicts aspects of a mobile computing device 2600D having a display screen 2610D with a graphical user interface 2620D. As shown here, user interface 2620D includes a presentation of a home screen that opens to “Profile”, “Reports”, “Points”, and “Calculate Dose” options.

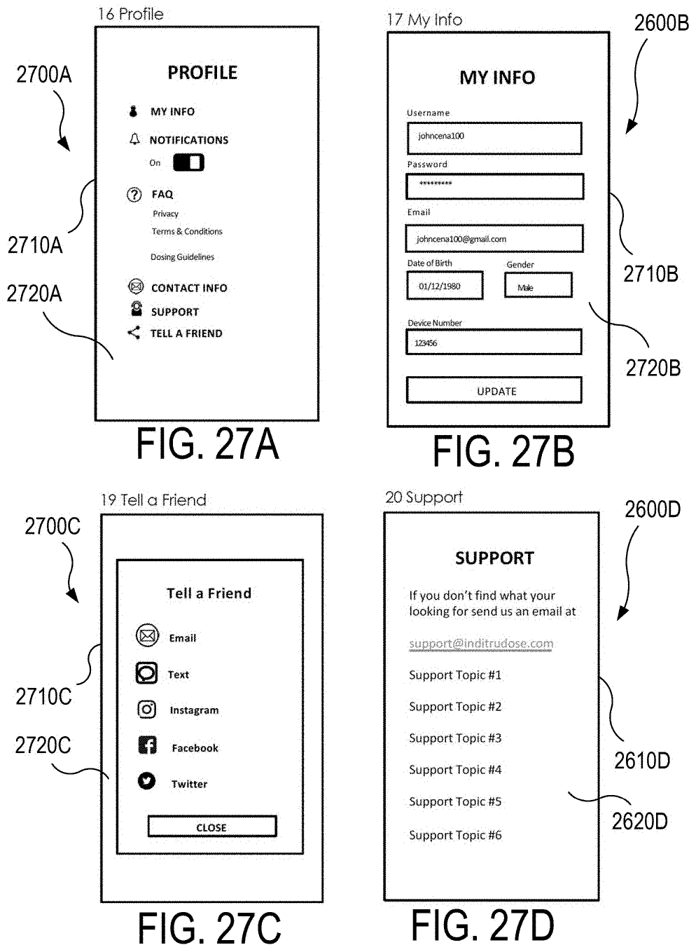

FIG. 27A depicts aspects of a mobile computing device 2700A having a display screen 2710A with a graphical user interface 2720A. As shown here, user interface 2720A includes a presentation of a profile screen that opens or provides access to “My Info”, “Notifications”, “FAQ”, “Contact Info”, “Support”, and “Calculate Dose” options.

FIG. 27B depicts aspects of a mobile computing device 2700B having a display screen 2710B with a graphical user interface 2720B. As shown here, user interface 2720B includes a presentation of a My Info screen.

FIG. 27C depicts aspects of a mobile computing device 2700C having a display screen 2710C with a graphical user interface 2720C. As shown here, user interface 2720C includes a presentation of a Tell A Friend screen.

FIG. 27D depicts aspects of a mobile computing device 2700D having a display screen 2710D with a graphical user interface 2720D. As shown here, user interface 2720D includes a presentation of a Support screen.

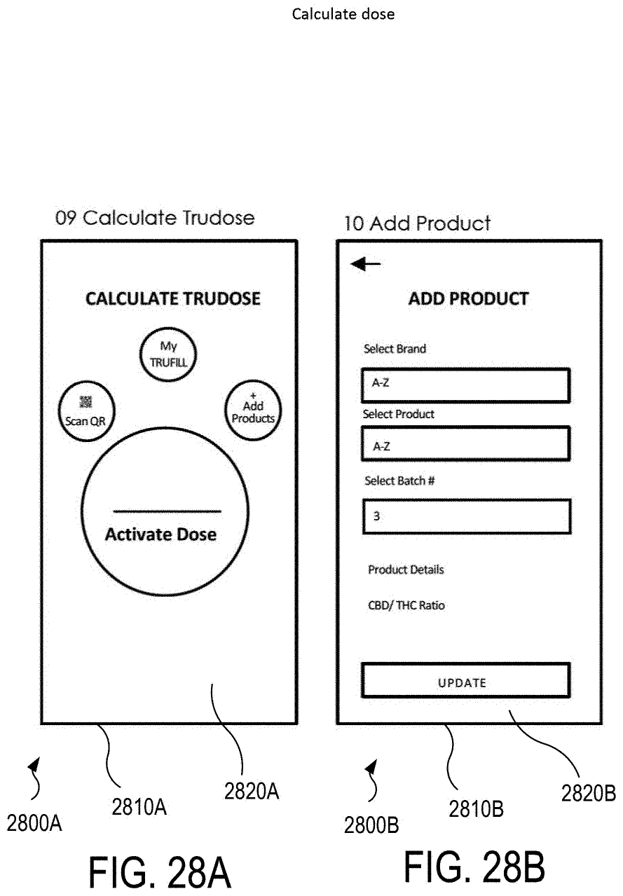

FIG. 28A depicts aspects of a mobile computing device 2800A having a display screen 2810A with a graphical user interface 2820A. As shown here, user interface 2820A includes a presentation of a Calculate Dose screen that opens or provides access to “Scan QR”, “My Fill”, “FAQ”, “Add Products”, and “Activate Dose” options.

FIG. 28B depicts aspects of a mobile computing device 2800B having a display screen 2810B with a graphical user interface 2820B. As shown here, user interface 2820B includes a presentation of an Add Product screen.

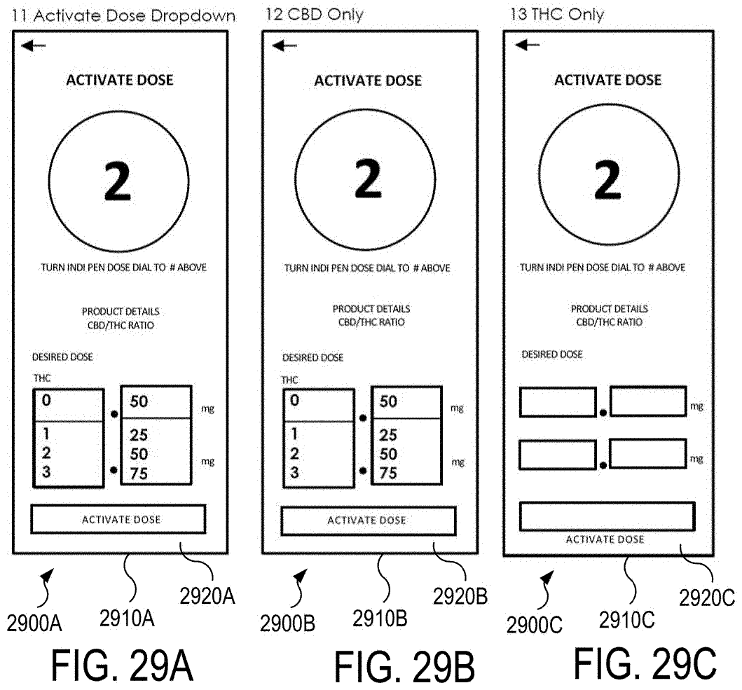

FIG. 29A depicts aspects of a mobile computing device 2900A having a display screen 2910A with a graphical user interface 2920A. As shown here, user interface 2920A includes a presentation of an Activate Dose Dropdown screen.

FIG. 29B depicts aspects of a mobile computing device 2900B having a display screen 2910B with a graphical user interface 2920B. As shown here, user interface 2920B includes a presentation of a CBD Only screen.

FIG. 29C depicts aspects of a mobile computing device 2900C having a display screen 2910C with a graphical user interface 2920C. As shown here, user interface 2920C includes a presentation of a THC Only screen.

According to some embodiments, when calculating a dose, the computing device can be configured to default to the desired dose of the psychoactive component (e.g. THC), and the amount of psychoactive component constituent can be used to manage any amounts of other non-psychoactive components (e.g. CBD). Hence, if a formulation includes both THC and CBD, the amount of dispensed preparation can be determined based on the amount of THC to be dispensed.

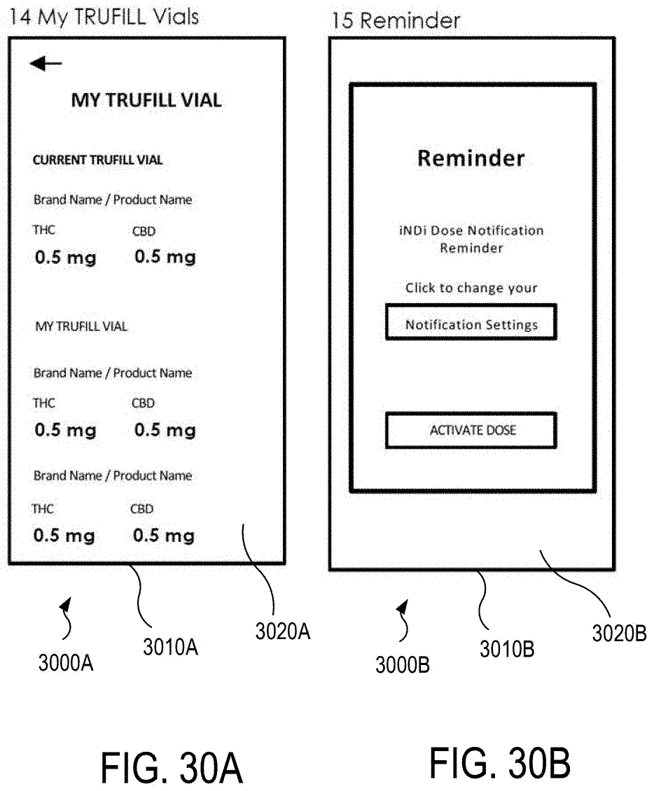

FIG. 30A depicts aspects of a mobile computing device 3000A having a display screen 3010A with a graphical user interface 3020A. As shown here, user interface 3020A includes a presentation of a My Fill Vials screen.

FIG. 30B depicts aspects of a mobile computing device 3000B having a display screen 3010B with a graphical user interface 3020B. As shown here, user interface 3020B includes a presentation of a Reminder screen.

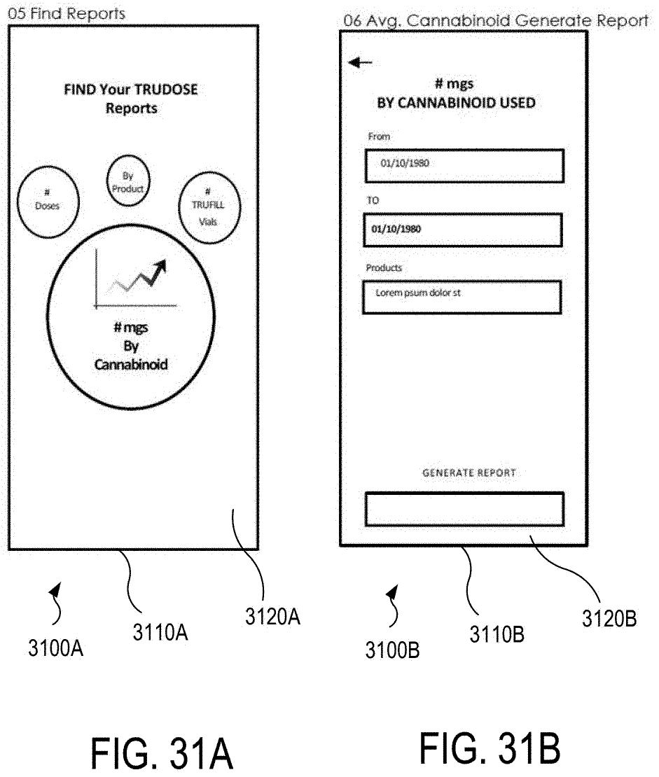

FIG. 31A depicts aspects of a mobile computing device 3100A having a display screen 3110A with a graphical user interface 3120A. As shown here, user interface 3120A includes a presentation of a Find Reports screen.

FIG. 31B depicts aspects of a mobile computing device 3100B having a display screen 3110B with a graphical user interface 3120B. As shown here, user interface 3120B includes a presentation of an Average Cannabinoid Generate Report screen.

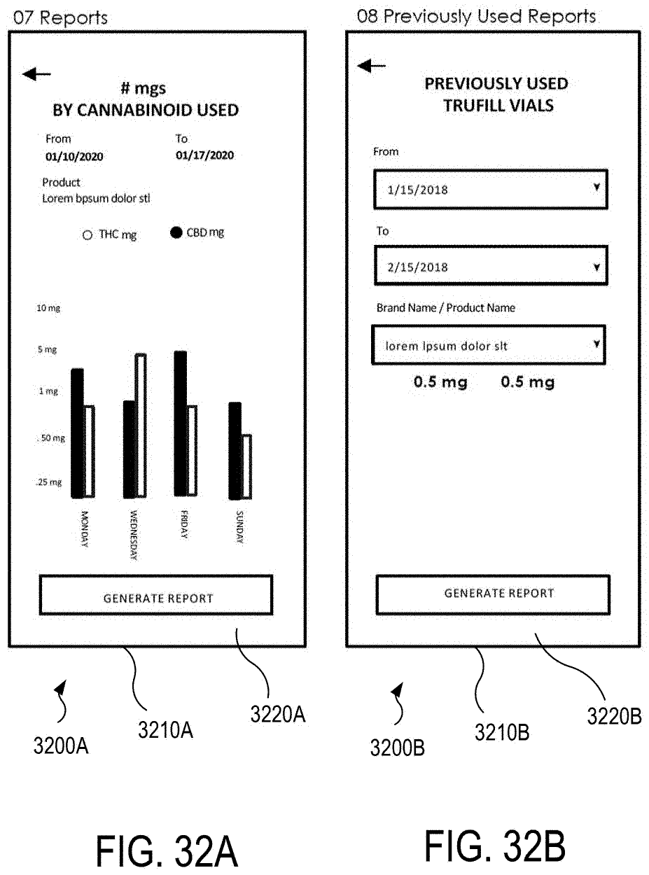

FIG. 32A depicts aspects of a mobile computing device 3200A having a display screen 3210A with a graphical user interface 3220A. As shown here, user interface 3220A includes a presentation of a Reports screen.

FIG. 32B depicts aspects of a mobile computing device 3200B having a display screen 3210B with a graphical user interface 3220B. As shown here, user interface 3220B includes a presentation of a Previously Used Reports screen.

Although the preceding description contains significant detail in relation to certain preferred embodiments, it should not be construed as limiting the scope of the invention but rather as providing illustrations of the preferred embodiments.

All features of the described systems and devices are applicable to the described methods mutatis mutandis, and vice versa. Embodiments of the present invention encompass kits having chambered dispensing systems as disclosed herein. In some embodiments, the kit includes one or more chambered dispensing systems, along with instructions for using the system for example according to any of the methods disclosed herein.

Although the foregoing invention has been described in some detail by way of illustration and example for purposes of clarity of understanding, one of skill in the art will appreciate that certain changes, modifications, alternate constructions, and/or equivalents may be practiced or employed as desired, and within the scope of the appended claims. In addition, each reference provided herein in incorporated by reference in its entirety to the same extent as if each reference was individually incorporated by reference. Relatedly, all publications, patents, patent applications, journal articles, books, technical references, and the like mentioned in this specification are herein incorporated by reference to the same extent as if each individual publication, patent, patent application, journal article, book, technical reference, or the like was specifically and individually indicated to be incorporated by reference.

1-20. (canceled)

21. A multi-chamber multi-cannabinoid medicament dispensing device, comprising:
a first medicament vial having a tip, and containing a first liquid medicament comprising a first cannabinoid, the first cannabinoid comprising a non-psychoactive Cannabis molecule;
a first plunger configured to dispense an amount of the first liquid medicament from the tip of the first medicament vial;
a second medicament vial having a tip, and containing a second liquid medicament comprising a second cannabinoid, the second cannabinoid comprising a psychoactive Cannabis molecule;
a second plunger configured to dispense an amount of the second liquid medicament from the tip of the second medicament vial;
a front housing;
a rear housing coupled with the front housing, wherein the coupled front housing and rear housing contain the first plunger and the second plunger; and
a dispensing spout coupled with the front housing and the rear housing, the dispensing spout comprising a first proximal aperture, a second proximal aperture, an interior chamber, and a distal aperture;
wherein the first medicament vial is positioned partially within the first proximal aperture of the dispensing spout and the second medicament vial is positioned partially within the second proximal aperture of the dispensing spout, and
wherein the tip of the first medicament vial is positioned within the interior chamber of the dispensing spout and the tip of the second medicament vial is positioned within the interior chamber of the dispensing spout, such that the amount of the first liquid medicament is dispensed from the tip of the first medicament vial into the interior chamber, and the amount of second liquid medicament is dispensed from the tip of the second medicament vial into the interior chamber to combine with the dispensed amount of the first liquid medicament within the interior chamber, and
wherein the distal aperture of the dispensing spout is configured to allow the combination of the dispensed amount of the first liquid medicament and the dispensed amount of the second liquid medicament to pass from the interior chamber of the dispensing spout therethrough.

22. The device of claim 21, further comprising a circuit board assembly, a first motor coupled with the first plunger, and a second motor coupled with the second plunger, wherein the circuit board assembly engages the first motor and the second motor.

23. The device of claim 22, wherein the front housing and the rear housing cover the circuit board assembly and leave the tip of the first medicament vial and the tip of the second medicament vial exposed, to facilitate removal of the first medicament vial and the second medicament vial from the multi-chamber multi-cannabinoid medicament dispensing device for replacement.

24. The device of claim 22, further comprising UP and DOWN keypad buttons for the first medicament vial operably connected to the circuit board assembly and UP and DOWN keypad buttons for the second medicament vial operably connected to the circuit board assembly, and wherein the UP and DOWN keypad buttons for the first medicament vial and the UP and DOWN keypad buttons for the second medicament vial enable selection of a desired relationship between a volume of the first liquid medicament containing the non-psychoactive Cannabis molecule and a volume of the second liquid medicament containing the psychoactive Cannabis molecule.

25. The device of claim 21, wherein the non-psychoactive Cannabis molecule is cannabidiol and the psychoactive Cannabis molecule is tetrahydrocannabinol.

26. The device of claim 21, further comprising a circuit board assembly that tracks the dispensed amount of the first liquid medicament and the dispensed amount of the second liquid medicament.

27. A multi-chamber multi-cannabinoid medicament dispensing device, comprising:
a first medicament vial having a tip, and containing a first liquid medicament comprising a first cannabinoid, the first cannabinoid comprising a non-psychoactive Cannabis molecule;
a first plunger configured to dispense an amount of the first liquid medicament from the tip of the first medicament vial;
a second medicament vial having a tip, and containing a second liquid medicament comprising a second cannabinoid, the second cannabinoid comprising a psychoactive Cannabis molecule;
a second plunger configured to dispense an amount of the second liquid medicament from the tip of the second medicament vial; and
a dispensing spout comprising a first proximal aperture, a second proximal aperture, an interior chamber, and a distal aperture;
wherein the first medicament vial is positioned partially within the first proximal aperture of the dispensing spout and the second medicament vial is positioned partially within the second proximal aperture of the dispensing spout, and
wherein the tip of the first medicament vial is positioned within the interior chamber of the dispensing spout and the tip of the second medicament vial is positioned within the interior chamber of the dispensing spout, such that the amount of the first liquid medicament is dispensed from the tip of the first medicament vial into the interior chamber, and the amount of second liquid medicament is dispensed from the tip of the second medicament vial into the interior chamber to combine with the dispensed amount of the first liquid medicament within the interior chamber, and
wherein the distal aperture of the dispensing spout is configured to allow the combination of the dispensed amount of the first liquid medicament and the dispensed amount of the second liquid medicament to pass from the interior chamber of the dispensing spout therethrough.

28. The device of claim 27, further comprising a circuit board assembly, a first motor coupled with the first plunger, and a second motor coupled with the second plunger, wherein the circuit board assembly engages the first motor and the second motor.

29. The device of claim 28, further comprising UP and DOWN keypad buttons for the first medicament vial operably connected to the circuit board assembly and UP and DOWN keypad buttons for the second medicament vial operably connected to the circuit board assembly, and wherein the UP and DOWN keypad buttons for the first medicament vial and the UP and DOWN keypad buttons for the second medicament vial enable selection of a desired relationship between a volume of the first liquid medicament containing the non-psychoactive Cannabis molecule and a volume of the second liquid medicament containing the psychoactive Cannabis molecule.

30. The device of claim 27, wherein the non-psychoactive Cannabis molecule is cannabidiol and the psychoactive Cannabis molecule is tetrahydrocannabinol.

31. The device of claim 27, further comprising a circuit board assembly that tracks the dispensed amount of the first liquid medicament and the dispensed amount of the second liquid medicament.

32. A multi-chamber dispensing device, comprising:
a first vial having a tip, and containing a first liquid;
a first plunger configured to dispense an amount of the first liquid from the tip of the first vial;
a second vial having a tip, and containing a second liquid;
a second plunger configured to dispense an amount of the second liquid from the tip of the second vial; and
a dispensing spout comprising a first proximal aperture, a second proximal aperture, an interior chamber, and a distal aperture;
wherein the first vial is positioned partially within the first proximal aperture of the dispensing spout and the second vial is positioned partially within the second proximal aperture of the dispensing spout, and
wherein the tip of the first vial is positioned within the interior chamber of the dispensing spout and the tip of the second vial is positioned within the interior chamber of the dispensing spout, such that the amount of the first liquid is dispensed from the tip of the first vial into the interior chamber, and the amount of second liquid is dispensed from the tip of the second vial into the interior chamber to combine with the dispensed amount of the first liquid within the interior chamber, and
wherein the distal aperture of the dispensing spout is configured to allow the combination of the dispensed amount of the first liquid and the dispensed amount of the second liquid to pass from the interior chamber of the dispensing spout therethrough.

33. The device of claim 32, further comprising a circuit board assembly, a first motor coupled with the first plunger, and a second motor coupled with the second plunger, wherein the circuit board assembly engages the first motor and the second motor.

34. The device of claim 33, further comprising UP and DOWN keypad buttons for the first vial operably connected to the circuit board assembly and UP and DOWN keypad buttons for the second vial operably connected to the circuit board assembly, and wherein the UP and DOWN keypad buttons for the first vial and the UP and DOWN keypad buttons for the second vial enable selection of a desired relationship between a volume of the first liquid and a volume of the second liquid.

35. The device of claim 32, wherein the first liquid comprises a non-psychoactive Cannabis molecule, and wherein the second liquid comprises a psychoactive Cannabis molecule.

36. The device of claim 35, wherein the non-psychoactive Cannabis molecule is cannabidiol and the psychoactive Cannabis molecule is tetrahydrocannabinol.

37. The device of claim 32, wherein the first liquid comprises a Cannabis molecule selected from the group consisting of a cannabinoid, a terpene, and a flavonoid, and wherein the second liquid comprises a Cannabis molecule selected from the group consisting of a cannabinoid, a terpene, and a flavonoid.

38. The device of claim 32, further comprising a circuit board assembly that tracks the dispensed amount of the first liquid and the dispensed amount of the second liquid.
